SEQUENCE LISTING (2 sequences)

SEQ ID NO: 1 - protein, 120 aa, synthetic construct
  REGION 1..120: note = Synthetic Construct
QVQLVQSGAEVKKPGASVKVSCKASGLTIEDYYMHWVRQAPGQGLEWMGWIDPENGDTEYGPKFQGRVTMTRDTSINTAYMELSRLRSDDTAVYYCAVHNAHYGTWFAYWGQGTLVTVSS

SEQ ID NO: 2 - protein, 113 aa, synthetic construct
  REGION 1..113: note = Synthetic Construct
DVVMTQSPLSLPVTLGQPASISCRSSQSLLHSSGNTYLEWYQQRPGQSPRPLIYKISTRFSGVPDRFSGSGSGTDFTLKISRVEAEDVGVYYCFQGSHVPYTFGGGTKVEIKR

(19) United States
(12) Patent Application Publication
(10) Pub. No.: US 2024/0158484 A1
(43) Pub. Date: May 16, 2024

(54) HUMANIZED ANTI-LIV1 ANTIBODIES FOR THE TREATMENT OF CANCER

(71) Applicant: Seagen Inc., Bothell, WA (US)

(72) Inventors: Matt ONSUM, Bothell, WA (US); Hailing LU, Bothell, WA (US); Zejing WANG, Bothell, WA (US)

(21) Appl. No.: 18/487,900

(22) Filed: Oct. 16, 2023

Related U.S. Application Data
(63) Continuation of application No. 17/449,074, filed on Sep. 27, 2021, now abandoned.
(60) Provisional application No. 63/084,444, filed on Sep. 28, 2020.
(60) Provisional application No. 63/243,025, filed on Sep. 10, 2021.

(51) Int. Cl. C07K 16/18; A61K 47/68; A61P 35/00; A61P 35/04; C07K 16/28

(52) U.S. Cl. CPC C07K 16/18; A61K 47/6817; A61K 47/6843; A61P 35/00; A61P 35/04; C07K 16/2818; A61K 2039/505

(57) ABSTRACT

Methods for using anti-LIV1 antibodies and antibody-drug conjugates, including anti-LIV1 antibody-drug conjugates, to inhibit proliferation of a cell, as well as for the treatment of cancers, such as, e.g., prostate cancer and melanoma, are provided.

### CROSS-REFERENCE TO RELATED APPLICATIONS

This application claims priority to U.S. Provisional Application No. 63/084,444, filed on Sep. 28, 2020, and U.S. Provisional Application No. 63/243,025, filed Sep. 10, 2021, the contents of each of which are incorporated herein by reference in their entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 761682004600SEQLIST.TXT, date recorded: Sep. 24, 2021, size: 2,442 bytes).

### TECHNICAL FIELD

The present invention relates to the field of antibody-based cancer therapeutics. In particular, the present invention relates to the use of humanized anti-LIV1 antibodies and antigen-binding fragments or antibody-drug conjugates thereof (e.g., LIV1-antibody-drug conjugates (LIV1-ADCs)) for the treatment of cancer, such as solid tumors, such as, e.g., locally advanced or metastatic solid tumors (e.g., prostate cancer and melanoma).

### BACKGROUND

LIV1 (SLC39A6) is a member of the solute carrier family, a multi-span transmembrane protein with putative zinc transporter and metalloproteinase activity. LIV1 was first identified as an estrogen-induced gene in the breast cancer cell line ZR-75-1. LIV1 is expressed in most subtypes of metastatic breast cancer.

Cancer remains to be one of the most deadly threats to human health. In the U.S., cancer affects nearly 1.3 million new patients each year, and is the second leading cause of death after heart disease, accounting for approximately 1 in 4 deaths. It is also predicted that cancer may surpass cardiovascular diseases as the number one cause of death within 5 years. Solid tumors are responsible for most of those deaths. Although there have been significant advances in the medical treatment of certain cancers, the overall 5-year survival rate for all cancers has improved only by about 10% in the past 20 years. Cancers, or malignant tumors, metastasize and grow rapidly in an uncontrolled manner, making timely detection and treatment extremely difficult.

Prostate cancer is a form of cancer that develops in the prostate gland. Globally, prostate cancer is the second-most common cancer. It is the fifth-leading cause of cancer-related death in men. In 2018, it was diagnosed in 1.2 million and caused 359,000 deaths. Prostate cancer cells can spread by breaking away from a prostate tumor. They can travel through blood vessels or lymph nodes to reach other parts of the body. After spreading, cancer cells may attach to other tissues and grow to form new tumors, causing damage where they land. Many cases are managed with active surveillance or watchful waiting. Other treatments may include a combination of surgery, radiation therapy, hormone therapy, or chemotherapy. The cancer chemotherapeutic docetaxel has been used as treatment for castration-resistant prostate cancer (CRPC) with a median survival benefit of 2 to 3 months. A second-line chemotherapy treatment is cabazitaxel. A combination of bevacizumab, docetaxel, thalidomide and prednisone appears effective in the treatment of CRPC. Immunotherapy treatment with sipuleucel-T in CRPC increases survival by four months. The second line hormonal therapy abiraterone increases survival by 4.6 months. Enzalutamide is another second line hormonal agent with a five month survival advantage. Both abiraterone and enzalutamide are currently in clinical trials in those with CRPC who have not previously received chemotherapy. More effective treatments for these patients with locally advanced or systemic disease are needed.

Melanoma, also known as malignant melanoma, is a type of skin cancer that develops from the pigment-producing cells known as melanocytes. Melanomas typically occur in the skin but may rarely occur in the mouth, intestines or eye (uveal melanoma). In women, they most commonly occur on the legs, while in men they most commonly occur on the back. About 25% of melanomas develop from moles. Changes in a mole that can indicate melanoma include an increase in size, irregular edges, change in color, itchiness or skin breakdown. Treatment is typically removal by surgery. In those with slightly larger cancers, nearby lymph nodes may be tested for spread (metastasis). Most people are cured if spread has not occurred. For those in whom melanoma has spread, immunotherapy, biologic therapy, radiation therapy or chemotherapy may improve survival. With treatment, the five-year survival rates in the United States are 99% among those with localized disease, 65% when the disease has spread to lymph nodes and 25% among those with distant spread. Melanoma is the most dangerous type of skin cancer. Globally, in 2012, it newly occurred in 232,000 people. In 2015, there were 3.1 million people with active disease, which resulted in 59,800 deaths. Australia and New Zealand have the highest rates of melanoma in the world. There are also high rates in Northern Europe and North America, while it is less common in Asia, Africa and Latin America. In the United States melanoma occurs about 1.6 times more often in men than women. There are four main types of melanoma, superficial spreading melanoma, nodular melanoma, lentigo maligna melanoma, and acral lentiginous melanoma. Chemotherapy drugs such as Dacarbazine have been the backbone of metastatic melanoma treatment since FDA approval in 1975 however, its efficacy in terms of survival has never been proven in a randomized controlled trial. Small-molecule targeted therapies are also used to treat melanoma. The main treatments are BRAF, C-Kit and NRAS inhibitors. BRAF inhibitors, such as vemurafenib and dabrafenib and a MEK inhibitor trametinib are the most effective, approved treatments for BRAF positive melanoma. The therapy combination of dabrafenib and trametinib has a 3-year PFS of 23%, and 5-year PFS of 13%.

There is clearly a significant need for effective treatments for solid tumors, particularly locally advanced or metastatic solid tumors, such as prostate cancer and melanoma. The present invention meets the need for improved treatment of solid tumors, such as, e.g., locally advanced or metastatic solid tumors (e.g., prostate cancer and melanoma) by providing a highly specific and effective anti-LIV1-antibody-drug conjugate.

All references cited herein, including patent applications, patent publications, and scientific literature, are herein incorporated by reference in their entirety, as if each individual reference were specifically and individually indicated to be incorporated by reference.

### SUMMARY

Provided herein is a method of treating a subject having or at risk of having cancer, comprising administering to the subject a therapeutically effective dose of an antibody or an antigen-binding fragment thereof that specifically binds human LIV1, wherein the antibody or antigen-binding fragment thereof comprises a heavy chain variable region (HCVR) having at least 95% identity to SEQ ID NO:1, and a light chain variable region (LCVR) having at least 95% identity to SEQ ID NO:2, wherein the cancer is prostate cancer. In some embodiments, the cancer is metastatic. In some embodiments, the prostate cancer is castration resistant prostate cancer. In some embodiments, the subject has not been previously treated with chemotherapy for the castration resistant prostate cancer. In some embodiments, the subject has not been previously treated with radioisotope therapy. In some embodiments, the subject has been previously treated with no more than one line of an androgen-receptor targeted therapy. In some embodiments, the androgen-receptor targeted therapy is selected from the group consisting of abiraterone acetate, enzalutamide, apalutamide, and darolutamide. In some embodiments, the subject does not have a BRCA mutation. In some embodiments, the prostate cancer is an adenocarcinoma of the prostate.

Also provided herein is a method of treating a subject having or at risk of having cancer, comprising administering to the subject a therapeutically effective dose of an antibody or an antigen-binding fragment thereof that specifically binds human LIV1, wherein the antibody or antigen-binding fragment thereof comprises a heavy chain variable region (HCVR) having at least 95% identity to SEQ ID NO:1, and a light chain variable region (LCVR) having at least 95% identity to SEQ ID NO:2, wherein the cancer is melanoma. In some embodiments, the cancer is locally advanced unresectable or metastatic. In some embodiments, the subject has not been previously treated with cytotoxic chemotherapy. In some embodiments, the subject has been previously treated with no more than 2 prior systemic therapies for advanced disease. In some embodiments, the melanoma is cutaneous malignant melanoma. In some embodiments, the subject has been previously treated with an anti-PD-L1 or anti-PD-1 therapy. In some embodiments, the subject was treated with ipilimumab in combination with the anti-PD-L1 or anti-PD-1 therapy. In some embodiments, the subject has a BRAF mutation. In some embodiments, the subject was treated with a BRAF inhibitor prior to being treated with the anti-PD-L1 or anti-PD-1 therapy. In some embodiments, the subject was treated with a MEK inhibitor in combination with the BRAF inhibitor.

In some embodiments, the heavy chain variable region of the antibody or antigen-binding fragment thereof comprises the three complementarity determining regions (CDRs) of SEQ ID NO:1 and the light chain variable region of the antibody or antigen-binding fragment thereof comprises the three CDRs of SEQ ID NO:2. In some embodiments, the heavy chain variable region has at least 98% identity to SEQ ID NO:1 and the light chain variable region has at least 98% identity to SEQ ID NO:2. In some embodiments, the heavy chain variable region has at least 99% identity to SEQ ID NO:1 and the light chain variable region has at least 99% identity to SEQ ID NO:2. In some embodiments, the heavy chain variable region comprises the sequence of SEQ ID NO:1 and the light chain variable region comprises the sequence of SEQ ID NO:2. In some embodiments, the antibody or antigen-binding fragment thereof is conjugated to monomethyl auristatin E (MMAE):

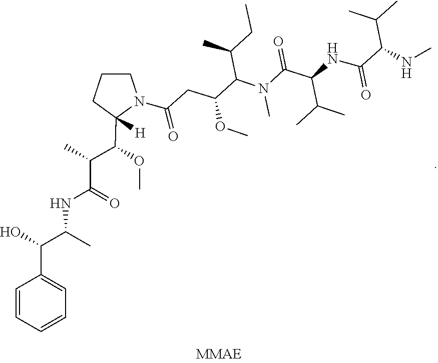 [SMILES: CC[C@H](C)C([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](NC)C(C)C)C(C)C]

In some embodiments, the antibody or antigen-binding fragment thereof is conjugated to valine-citrulline-monomethyl auristatin E (vcMMAE):

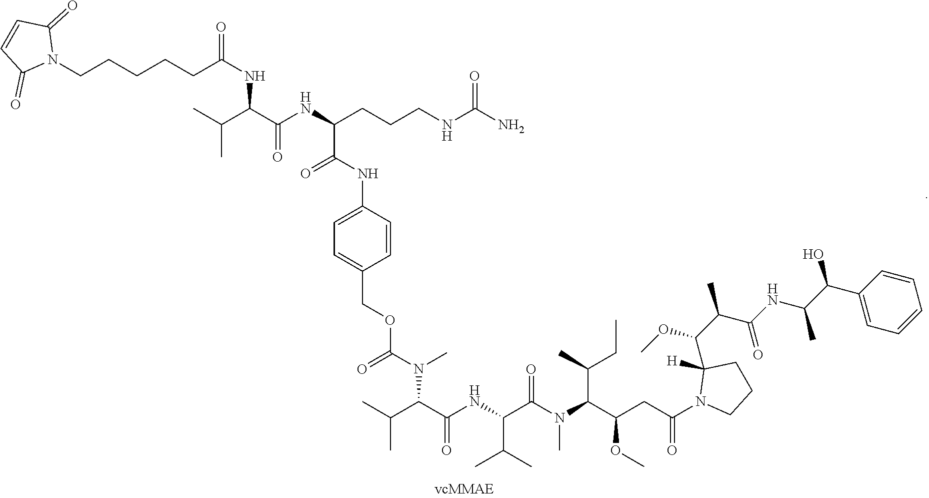 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](NC(=O)CCCCCN2C(=O)C=CC2=O)C(C)C)cc1)C(C)C]

In some embodiments, a vcMMAE to antibody or antigen-binding fragment thereof ratio is from about 1 to about 8. In some embodiments, the vcMMAE to antibody or antigen-binding fragment thereof ratio is about 4. In some embodiments, the dose administered is less than about 200 mg of the antibody or antigen-binding fragment thereof per treatment cycle. In some embodiments, the dose is about 1.0 mg/kg of body weight of the subject. In some embodiments, the dose administered is less than about 100 mg of the antibody or antigen-binding fragment thereof per treatment cycle. In some embodiments, the dose is about 1.25 mg/kg of body weight of the subject. In some embodiments, the dose administered is less than about 125 mg of the antibody or antigen-binding fragment thereof per treatment cycle. In some embodiments, the treatment cycle is a Q1W treatment cycle. In some embodiments, In some embodiments, the subject has been previously treated with one or more therapeutic agents and did not respond to the treatment, wherein the one or more therapeutic agents is not the antibody or antigen-binding fragment thereof. In some embodiments, the subject has been previously treated with one or more therapeutic agents and relapsed after the treatment, wherein the one or more therapeutic agents is not the antibody or antigen-binding fragment thereof. In some embodiments, the subject has been previously treated with one or more therapeutic agents and has experienced disease progression during treatment, wherein the one or more therapeutic agents is not the antibody or antigen-binding fragment thereof. In some embodiments, the cancer is an advanced stage cancer. In some embodiments, the advanced stage cancer is a stage 3 or stage 4 cancer. In some embodiments, the cancer is recurrent cancer. In some embodiments, the cancer is unresectable. In some embodiments, the subject received prior treatment with standard of care therapy for the cancer and failed the prior treatment. In some embodiments, at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of the cancer cells express LIV1. In some embodiments, one or more therapeutic effects in the subject is improved after administration of the antibody or antigen-binding fragment thereof relative to a baseline. In some embodiments, the one or more therapeutic effects is selected from the group consisting of: size of a tumor derived from the cancer, objective response rate, duration of response, time to response, progression free survival, overall survival, prostate-specific antigen (PSA) level, PSA duration of response, and PSA-PFS. In some embodiments, the size of a tumor derived from the cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of the antibody or antigen-binding fragment thereof. In some embodiments, the objective response rate is at least about 20%, at least about 25%, at least about 30%, at least about 35%, at least about 40%, at least about 45%, at least about 50%, at least about 60%, at least about 70%, or at least about 80%. In some embodiments, the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody or antigen-binding fragment thereof. In some embodiments, the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody or antigen-binding fragment thereof. In some embodiments, the duration of response to the antibody-drug conjugate is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody or antigen-binding fragment thereof. In some embodiments, the cancer is prostate cancer and the subject's PSA level is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the subject's PSA level before administration of the antibody or antigen-binding fragment thereof. In some embodiments, the cancer is prostate cancer and the subject exhibits PSA progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody or antigen-binding fragment thereof. In some embodiments, the cancer is prostate cancer and the PSA duration of response to the antibody-drug conjugate is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody or antigen-binding fragment thereof. In some embodiments, the subject has one or more adverse events and is further administered an additional therapeutic agent to eliminate or reduce the severity of the one or more adverse events. In some embodiments, the subject is at risk of developing one or more adverse events and is further administered an additional therapeutic agent to prevent or reduce the severity of the one or more adverse events. In some embodiments, the one or more adverse events is a grade 3 or greater adverse event. In some embodiments, the one or more adverse events is a serious adverse event. In some embodiments, the route of administration for the antibody or antigen-binding fragment thereof is intravenous infusion. In some embodiments, the antibody or antigen-binding fragment thereof is administered as a monotherapy. In some embodiments, the antibody or antigen-binding fragment thereof is administered in combination with a checkpoint inhibitor. In some embodiments, the checkpoint inhibitor is an anti-PD-1 antibody, an anti-PD-L1 antibody, an anti-CTLA4 antibody, B7-DC-Fc, LAG3, or TIM3. In some embodiments, the checkpoint inhibitor is selected from the group consisting of MEDI0680, AMP-224, nivolumab, pembrolizumab, pidilizumab, MEDI4736, MPDL3280A, ipilimumab and tremelimumab. In some embodiments, the checkpoint inhibitor is pembrolizumab. In some embodiments, the antibody or antigen-binding fragment thereof is in a pharmaceutical composition comprising the antibody or antigen-binding fragment thereof and a pharmaceutically acceptable carrier. In some embodiments, the subject is a human.

Also provided herein is a kit comprising a dosage ranging from about 0.5 mg/kg to about 2.8 mg/kg of an antibody or antigen-binding fragment thereof that binds LIV1 and instructions for using the antibody or antigen-binding fragment thereof according to any of the embodiments herein.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### DETAILED DESCRIPTION

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

### I. DEFINITIONS

As used herein, including the appended claims, the singular forms of words such as “a,” “an,” and “the,” include their corresponding plural references unless the context clearly dictates otherwise.

An “antibody-drug conjugate” or “ADC” refers to an antibody conjugated to a cytotoxic agent or cytostatic agent. Typically, antibody-drug conjugates bind to a target antigen (e.g., LIV1) on a cell surface, followed by internalization of the antibody-drug conjugate into the cell and subsequent release of the drug into the cell. In certain exemplary embodiments, an antibody-drug conjugate is a LIV1-ADC.

A “polypeptide” or “polypeptide chain” is a polymer of amino acid residues joined by peptide bonds, whether produced naturally or synthetically. Polypeptides of less than about 10 amino acid residues are commonly referred to as “peptides.”

A “protein” is a macromolecule comprising one or more polypeptide chains. A protein may also comprise non-peptidic components, such as carbohydrate groups. Carbohydrates and other non-peptidic substituents may be added to a protein by the cell in which the protein is produced, and will vary with the type of cell. Proteins are defined herein in terms of their amino acid backbone structures. Substituents such as carbohydrate groups are generally not specified, but may be present nonetheless.

The terms “amino-terminal” and “carboxy-terminal” denote positions within polypeptides. Where the context allows, these terms are used with reference to a particular sequence or portion of a polypeptide to denote proximity or relative position. For example, a certain sequence positioned carboxy-terminal to a reference sequence within a polypeptide is located proximal to the carboxy terminus of the reference sequence, but is not necessarily at the carboxy terminus of the complete polypeptide.

For purposes of classifying amino acids substitutions as conservative or nonconservative, the following amino acid substitutions are considered conservative substitutions: serine substituted by threonine, alanine, or asparagine; threonine substituted by proline or serine; asparagine substituted by aspartic acid, histidine, or serine; aspartic acid substituted by glutamic acid or asparagine; glutamic acid substituted by glutamine, lysine, or aspartic acid; glutamine substituted by arginine, lysine, or glutamic acid; histidine substituted by tyrosine or asparagine; arginine substituted by lysine or glutamine; methionine substituted by isoleucine, leucine or valine; isoleucine substituted by leucine, valine, or methionine; leucine substituted by valine, isoleucine, or methionine; phenylalanine substituted by tyrosine or tryptophan; tyrosine substituted by tryptophan, histidine, or phenylalanine; proline substituted by threonine; alanine substituted by serine; lysine substituted by glutamic acid, glutamine, or arginine; valine substituted by methionine, isoleucine, or leucine; and tryptophan substituted by phenylalanine or tyrosine. Conservative substitutions can also mean substitutions between amino acids in the same class. Classes are as follows: Group I (hydrophobic side chains): Met, Ala, Val, Leu, Ile; Group II (neutral hydrophilic side chains): Cys, Ser, Thr; Group III (acidic side chains): Asp, Glu; Group IV (basic side chains): Asn, Gln, His, Lys, Arg; Group V (residues influencing chain orientation): Gly, Pro; and Group VI (aromatic side chains): Trp, Tyr, Phe.

Two amino acid sequences have “100% amino acid sequence identity” if the amino acid residues of the two amino acid sequences are the same when aligned for maximal correspondence. Sequence comparisons can be performed using standard software programs such as those included in the LASERGENE bioinformatics computing suite, which is produced by DNASTAR (Madison, Wisconsin). Other methods for comparing two nucleotide or amino acid sequences by determining optimal alignment are well-known to those of skill in the art. (See, e.g., Peruski and Peruski, The Internet and the New Biology: Tools for Genomic and Molecular Research (ASM Press, Inc. 1997); Wu et al. (eds.), “Information Superhighway and Computer Databases of Nucleic Acids and Proteins,” in Methods in Gene Biotechnology 123-151 (CRC Press, Inc. 1997); Bishop (ed.), Guide to Human Genome Computing (2nd ed., Academic Press, Inc. 1998).) Two amino acid sequences are considered to have “substantial sequence identity” if the two sequences have at least about 80%, at least about 85%, at about least 90%, or at least about 95% sequence identity relative to each other.

Percentage sequence identities are determined with antibody sequences maximally aligned by the Kabat numbering convention. After alignment, if a subject antibody region (e.g., the entire variable domain of a heavy or light chain) is being compared with the same region of a reference antibody, the percentage sequence identity between the subject and reference antibody regions is the number of positions occupied by the same amino acid in both the subject and reference antibody region divided by the total number of aligned positions of the two regions, with gaps not counted, multiplied by 100 to convert to percentage.

Compositions or methods “comprising” one or more recited elements may include other elements not specifically recited. For example, a composition that comprises antibody may contain the antibody alone or in combination with other ingredients.

Designation of a range of values includes all integers within or defining the range.

In antibodies or other proteins described herein, reference to amino acid residues corresponding to those specified by SEQ ID NO includes post-translational modifications of such residues.

The term “antibody” denotes immunoglobulin proteins produced by the body in response to the presence of an antigen and that bind to the antigen, as well as antigen-binding fragments and engineered variants thereof. Hence, the term “antibody” includes, for example, intact monoclonal antibodies (e.g., antibodies produced using hybridoma technology) and antigen-binding antibody fragments, such as a F(ab′)₂, a Fv fragment, a diabody, a single-chain antibody, an scFv fragment, or an scFv-Fc. Genetically, engineered intact antibodies and fragments such as chimeric antibodies, humanized antibodies, single-chain Fv fragments, single-chain antibodies, diabodies, minibodies, linear antibodies, multivalent or multi-specific (e.g., bispecific) hybrid antibodies, and the like, are also included. Thus, the term “antibody” is used expansively to include any protein that comprises an antigen-binding site of an antibody and is capable of specifically binding to its antigen.

The term antibody or antigen-binding fragment thereof includes a “conjugated” antibody or antigen-binding fragment thereof or an “antibody-drug conjugate (ADC)” in which an antibody or antigen-binding fragment thereof is covalently or non-covalently bound to a pharmaceutical agent, e.g., to a cytostatic or cytotoxic drug.

The term “genetically engineered antibodies” refers to an antibody in which the amino acid sequence has been varied from that of the native or parental antibody. The possible variations are many, and range from the changing of just one or a few amino acids to the complete redesign of, for example, the variable or constant region. Changes in the constant region are, in general, made to improve or alter characteristics such as, e.g., complement binding and other effector functions. Typically, changes in the variable region are made to improve antigen-binding characteristics, improve variable region stability, and/or reduce the risk of immunogenicity.

The term “chimeric antibody” refers to an antibody in which a portion of the heavy and/or light chain is identical with or homologous to corresponding sequences in an antibody derived from a particular species (e.g., human) or belonging to a particular antibody class or subclass, while the remainder of the chain(s) is identical with or homologous to corresponding sequences in an antibody derived from another species (e.g., mouse) or belonging to another antibody class or subclass, as well as fragments of such antibodies, so long as they exhibit the desired biological activity.

An “antigen-binding site of an antibody” is that portion of an antibody that is sufficient to bind to its antigen. The minimum such region is typically a variable domain or a genetically engineered variant thereof. Single domain binding sites can be generated from camelid antibodies (see Muyldermans and Lauwereys, Mol. Recog. 12: 131-140, 1999; Nguyen et al., EMBO J. 19:921-930, 2000) or from VH domains of other species to produce single-domain antibodies (“dAbs,” see Ward et al., Nature 341: 544-546, 1989; U.S. Pat. No. 6,248,516 to Winter et al). Commonly, an antigen-binding site of an antibody comprises both a heavy chain variable (VH) domain and a light chain variable (VL) domain that bind to a common epitope. Within the context of the present invention, an antibody may include one or more components in addition to an antigen-binding site, such as, for example, a second antigen-binding site of an antibody (which may bind to the same or a different epitope or to the same or a different antigen), a peptide linker, an immunoglobulin constant region, an immunoglobulin hinge, an amphipathic helix (see Pack and Pluckthun, Biochem. 31: 1579-1584, 1992), a non-peptide linker, an oligonucleotide (see Chaudri et al., FEBS Letters 450:23-26, 1999), a cytostatic or cytotoxic drug, and the like, and may be a monomeric or multimeric protein. Examples of molecules comprising an antigen-binding site of an antibody are known in the art and include, for example, Fv, single-chain Fv (scFv), Fab, Fab′, F(ab′)2, F(ab)c, diabodies, minibodies, nanobodies, Fab-scFv fusions, bispecific (scFv)4-IgG, and bispecific (scFv)2-Fab. (See, e.g., Hu et al, Cancer Res. 56:3055-3061, 1996; Atwell et al., Molecular Immunology 33: 1301-1312, 1996; Carter and Merchant, Curr. Op. Biotechnol. 8:449-454, 1997; Zuo et al., Protein Engineering 13:361-367, 2000; and Lu et al., J. Immunol. Methods 267:213-226, 2002.)

The term “immunoglobulin” refers to a protein consisting of one or more polypeptides substantially encoded by immunoglobulin gene(s). One form of immunoglobulin constitutes the basic structural unit of native (i.e., natural or parental) antibodies in vertebrates. This form is a tetramer and consists of two identical pairs of immunoglobulin chains, each pair having one light chain and one heavy chain. In each pair, the light and heavy chain variable regions (VL and VH) are together primarily responsible for binding to an antigen, and the constant regions are primarily responsible for the antibody effector functions. Five classes of immunoglobulin protein (IgG, IgA, IgM, IgD, and IgE) have been identified in higher vertebrates. IgG comprises the major class, and it normally exists as the second most abundant protein found in plasma. In humans, IgG consists of four subclasses, designated IgG1, IgG2, IgG3, and IgG4. Each immunoglobulin heavy chain possesses a constant region that consists of constant region protein domains (CH1, hinge, CH2, and CH3; IgG3 also contains a CH4 domain) that are essentially invariant for a given subclass in a species.

DNA sequences encoding human and non-human immunoglobulin chains are known in the art. (See, e.g., Ellison et al, DNA 1: 11-18, 1981; Ellison et al, Nucleic Acids Res. 10:4071-4079, 1982; Kenten et al., Proc. Natl. Acad. Set USA 79:6661-6665, 1982; Seno et al., Nucl. Acids Res. 11:719-726, 1983; Riechmann et al., Nature 332:323-327, 1988; Amster et al., Nucl. Acids Res. 8:2055-2065, 1980; Rusconi and Kohler, Nature 314:330-334, 1985; Boss et al., Nucl. Acids Res. 12:3791-3806, 1984; Bothwell et al., Nature 298:380-382, 1982; van der Loo et al., Immunogenetics 42:333-341, 1995; Karlin et al., J. Mol. Evol. 22: 195-208, 1985; Kindsvogel et al., DNA 1:335-343, 1982; Breiner et al., Gene 18: 165-174, 1982; Kondo et al., Eur. J. Immunol. 23:245-249, 1993; and GenBank Accession No. J00228.) For a review of immunoglobulin structure and function see Putnam, The Plasma Proteins, Vol V, Academic Press, Inc., 49-140, 1987; and Padlan, Mol. Immunol. 31: 169-217, 1994. The term “immunoglobulin” is used herein for its common meaning, denoting an intact antibody, its component chains, or fragments of chains, depending on the context.

Full-length immunoglobulin “light chains” (about 25 kDa or 214 amino acids) are encoded by a variable region gene at the amino-terminus (encoding about 110 amino acids) and a by a kappa or lambda constant region gene at the carboxyl-terminus. Full-length immunoglobulin “heavy chains” (about 50 kDa or 446 amino acids) are encoded by a variable region gene (encoding about 116 amino acids) and a gamma, mu, alpha, delta, or epsilon constant region gene (encoding about 330 amino acids), the latter defining the antibody's isotype as IgG, IgM, IgA, IgD, or IgE, respectively. Within light and heavy chains, the variable and constant regions are joined by a “J” region of about 12 or more amino acids, with the heavy chain also including a “D” region of about 10 more amino acids. (See generally Fundamental Immunology (Paul, ed., Raven Press, N.Y., 2nd ed. 1989), Ch. 7).

An immunoglobulin light or heavy chain variable region (also referred to herein as a “light chain variable domain” (“VL domain”) or “heavy chain variable domain” (“VH domain”), respectively) consists of a “framework” region interrupted by three “complementarity determining regions” or “CDRs.” The framework regions serve to align the CDRs for specific binding to an epitope of an antigen. Thus, the term “CDR” refers to the amino acid residues of an antibody that are primarily responsible for antigen binding. From amino-terminus to carboxyl-terminus, both VL and VH domains comprise the following framework (FR) and CDR regions: FR1, CDR1, FR2, CDR2, FR3, CDR3, FR4.

The assignment of amino acids to each variable region domain is in accordance with the definitions of Kabat, Sequences of Proteins of Immunological Interest (National Institutes of Health, Bethesda, M D, 1987 and 1991). Kabat also provides a widely used numbering convention (Kabat numbering) in which corresponding residues between different heavy chain variable regions or between different light chain variable regions are assigned the same number. CDRs 1, 2 and 3 of a VL domain are also referred to herein, respectively, as CDR-L1, CDR-L2 and CDR-L3. CDRs 1, 2 and 3 of a VH domain are also referred to herein, respectively, as CDR-H1, CDR-H2 and CDR-H3. If so noted, the assignment of CDRs can be in accordance with IMGT® (Lefranc et al., Developmental & Comparative Immunology 27:55-77; 2003) in lieu of Kabat.

Numbering of the heavy chain constant region is via the EU index as set forth in Kabat (Kabat, Sequences of Proteins of Immunological Interest, National Institutes of Health, Bethesda, M D, 1987 and 1991).

Unless the context dictates otherwise, the term “monoclonal antibody” is not limited to antibodies produced through hybridoma technology. The term “monoclonal antibody” can include an antibody that is derived from a single clone, including any eukaryotic, prokaryotic or phage clone. In particular embodiments, the antibodies described herein are monoclonal antibodies.

The term “humanized VH domain” or “humanized VL domain” refers to an immunoglobulin VH or VL domain comprising some or all CDRs entirely or substantially from a non-human donor immunoglobulin (e.g., a mouse or rat) and variable domain framework sequences entirely or substantially from human immunoglobulin sequences. The non-human immunoglobulin providing the CDRs is called the “donor” and the human immunoglobulin providing the framework is called the “acceptor.” In some instances, humanized antibodies will retain some non-human residues within the human variable domain framework regions to enhance proper binding characteristics (e.g., mutations in the frameworks may be required to preserve binding affinity when an antibody is humanized).

A “humanized antibody” is an antibody comprising one or both of a humanized VH domain and a humanized VL domain. Immunoglobulin constant region(s) need not be present, but if they are, they are entirely or substantially from human immunoglobulin constant regions.

A humanized antibody is a genetically engineered antibody in which the CDRs from a non-human “donor” antibody are grafted into human “acceptor” antibody sequences (see, e.g., Queen, U.S. Pat. Nos. 5,530,101 and 5,585,089; Winter, U.S. Pat. No. 5,225,539; Carter, U.S. Pat. No. 6,407,213; Adair, U.S. Pat. No. 5,859,205; and Foote, U.S. Pat. No. 6,881,557). The acceptor antibody sequences can be, for example, a mature human antibody sequence, a composite of such sequences, a consensus sequence of human antibody sequences, or a germline region sequence.

Human acceptor sequences can be selected for a high degree of sequence identity in the variable region frameworks with donor sequences to match canonical forms between acceptor and donor CDRs among other criteria. Thus, a humanized antibody is an antibody having CDRs entirely or substantially from a donor antibody and variable region framework sequences and constant regions, if present, entirely or substantially from human antibody sequences. Similarly, a humanized heavy chain typically has all three CDRs entirely or substantially from a donor antibody heavy chain, and a heavy chain variable region framework sequence and heavy chain constant region, if present, substantially from human heavy chain variable region framework and constant region sequences. Similarly, a humanized light chain typically has all three CDRs entirely or substantially from a donor antibody light chain, and a light chain variable region framework sequence and light chain constant region, if present, substantially from human light chain variable region framework and constant region sequences.

A CDR in a humanized antibody is substantially from a corresponding CDR in a non-human antibody when at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% of corresponding residues (as defined by Kabat numbering), or wherein about 100% of corresponding residues (as defined by Kabat numbering), are identical between the respective CDRs. The variable region framework sequences of an antibody chain or the constant region of an antibody chain are substantially from a human variable region framework sequence or human constant region respectively when at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% of corresponding residues (as defined by Kabat numbering for the variable region and EU numbering for the constant region), or about 100% of corresponding residues (as defined by Kabat numbering for the variable region and EU numbering for the constant region) are identical.

Although humanized antibodies often incorporate all six CDRs (preferably as defined by Kabat or IMGT®) from a mouse antibody, they can also be made with fewer than all six CDRs (e.g., at least 3, 4, or 5) CDRs from a mouse antibody (e.g., Pascalis et al., J. Immunol. 169:3076, 2002; Vajdos et al., Journal of Molecular Biology, 320: 415-428, 2002; Iwahashi et al., Mol. Immunol. 36:1079-1091, 1999; Tamura et al, Journal of Immunology, 164: 1432-1441, 2000).

A CDR in a humanized antibody is “substantially from” a corresponding CDR in a non-human antibody when at least 60%, at least 85%, at least 90%, at least 95% or 100% of corresponding residues (as defined by Kabat (or IMGT)) are identical between the respective CDRs. In particular variations of a humanized VH or VL domain in which CDRs are substantially from a non-human immunoglobulin, the CDRs of the humanized VH or VL domain have no more than six (e.g., no more than five, no more than four, no more than three, no more than two, or nor more than one) amino acid substitutions (preferably conservative substitutions) across all three CDRs relative to the corresponding non-human VH or VL CDRs. The variable region framework sequences of an antibody VH or VL domain or, if present, a sequence of an immunoglobulin constant region, are “substantially from” a human VH or VL framework sequence or human constant region, respectively, when at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% of corresponding residues (as defined by Kabat numbering for the variable region and EU numbering for the constant region), or about 100% of corresponding residues (as defined by Kabat numbering for the variable region and EU numbering for the constant region) are identical. Hence, all parts of a humanized antibody, except the CDRs, are typically entirely or substantially from corresponding parts of natural human immunoglobulin sequences.

Antibodies are typically provided in isolated form. This means that an antibody is typically at least about 50% w/w pure of interfering proteins and other contaminants arising from its production or purification but does not exclude the possibility that the antibody is combined with an excess of pharmaceutical acceptable carrier(s) or other vehicle intended to facilitate its use. Sometimes antibodies are at least about 60%, about 70%, about 80%, about 90%, about 95% or about 99% w/w pure of interfering proteins and contaminants from production or purification. Antibodies, including isolated antibodies, can be conjugated to cytotoxic agents and provided as antibody drug conjugates.

Specific binding of an antibody to its target antigen typically refers an affinity of at least about 10⁶, about 10⁷, about 10⁸, about 10⁹, or about 10¹⁰ M⁻¹. Specific binding is detectably higher in magnitude and distinguishable from non-specific binding occurring to at least one non-specific target. Specific binding can be the result of formation of bonds between particular functional groups or particular spatial fit (e.g., lock and key type), whereas nonspecific binding is typically the result of van der Waals forces.

The term “epitope” refers to a site of an antigen to which an antibody binds. An epitope can be formed from contiguous amino acids or noncontiguous amino acids juxtaposed by tertiary folding of one or more proteins. Epitopes formed from contiguous amino acids are typically retained upon exposure to denaturing agents, e.g., solvents, whereas epitopes formed by tertiary folding are typically lost upon treatment with denaturing agents, e.g., solvents. An epitope typically includes at least about 3, and more usually, at least about 5, at least about 6, at least about 7, or about 8-10 amino acids in a unique spatial conformation. Methods of determining spatial conformation of epitopes include, for example, x-ray crystallography and two-dimensional nuclear magnetic resonance. See, e.g., Epitope Mapping Protocols, in Methods in Molecular Biology, Vol. 66, Glenn E. Morris, Ed. (1996).

Antibodies that recognize the same or overlapping epitopes can be identified in a simple immunoassay showing the ability of one antibody to compete with the binding of another antibody to a target antigen. The epitope of an antibody can also be defined by X-ray crystallography of the antibody bound to its antigen to identify contact residues.

Alternatively, two antibodies have the same epitope if all amino acid mutations in the antigen that reduce or eliminate binding of one antibody reduce or eliminate binding of the other (provided that such mutations do not produce a global alteration in antigen structure). Two antibodies have overlapping epitopes if some amino acid mutations that reduce or eliminate binding of one antibody reduce or eliminate binding of the other antibody.

Competition between antibodies can be determined by an assay in which a test antibody inhibits specific binding of a reference antibody to a common antigen (see, e.g., Junghans et al., Cancer Res. 50: 1495, 1990). A test antibody competes with a reference antibody if an excess of a test antibody inhibits binding of the reference antibody.

Antibodies identified by competition assay (competing antibodies) include antibodies that bind to the same epitope as the reference antibody and antibodies that bind to an adjacent epitope sufficiently proximal to the epitope bound by the reference antibody for steric hindrance to occur. Antibodies identified by a competition assay also include those that indirectly compete with a reference antibody by causing a conformational change in the target protein thereby preventing binding of the reference antibody to a different epitope than that bound by the test antibody.

An antibody effector function refers to a function contributed by an Fc region of an Ig. Such functions can be, for example, antibody-dependent cellular cytotoxicity (ADCC), antibody-dependent cellular phagocytosis (ADCP), or complement-dependent cytotoxicity (CDC). Such function can be affected by, for example, binding of an Fc region to an Fc receptor on an immune cell with phagocytic or lytic activity or by binding of an Fc region to components of the complement system. Typically, the effect(s) mediated by the Fc-binding cells or complement components result in inhibition and/or depletion of the LIV1-targeted cell. Fc regions of antibodies can recruit Fc receptor (FcR)-expressing cells and juxtapose them with antibody-coated target cells. Cells expressing surface FcR for IgGs including FcγRIII (CD16), FcγRII (CD32) and FcγRI (CD64) can act as effector cells for the destruction of IgG-coated cells. Such effector cells include monocytes, macrophages, natural killer (NK) cells, neutrophils and eosinophils. Engagement of FcγR by IgG activates ADCC or ADCP. ADCC is mediated by CD16+ effector cells through the secretion of membrane pore-forming proteins and proteases, while phagocytosis is mediated by CD32+ and CD64+ effector cells (see Fundamental Immunology, 4^{th} ed., Paul ed., Lippincott-Raven, N.Y., 1997, Chapters 3, 17 and 30; Uchida et al., J. Exp. Med. 199:1659-69, 2004; Akewanlop et al., Cancer Res. 61:4061-65, 2001; Watanabe et al., Breast Cancer Res. Treat. 53: 199-207, 1999).

In addition to ADCC and ADCP, Fc regions of cell-bound antibodies can also activate the complement classical pathway to elicit CDC. C1q of the complement system binds to the Fc regions of antibodies when they are complexed with antigens. Binding of C1q to cell-bound antibodies can initiate a cascade of events involving the proteolytic activation of C4 and C2 to generate the C3 convertase. Cleavage of C3 to C3b by C3 convertase enables the activation of terminal complement components including C5b, C6, C7, C8 and C9. Collectively, these proteins form membrane-attack complex pores on the antibody-coated cells. These pores disrupt the cell membrane integrity, killing the target cell (see Immunobiology, 6^{th} ed., Janeway et al, Garland Science, N. Y., 2005, Chapter 2).

The term “antibody-dependent cellular cytotoxicity” or “ADCC” refers to a mechanism for inducing cell death that depends on the interaction of antibody-coated target cells with immune cells possessing lytic activity (also referred to as effector cells). Such effector cells include natural killer cells, monocytes/macrophages and neutrophils. The effector cells attach to an Fc region of Ig bound to target cells via their antigen-combining sites. Death of the antibody-coated target cell occurs as a result of effector cell activity. In certain exemplary embodiments, an anti-LIV1 IgG1 antibody of the invention mediates equal or increased ADCC relative to a parental antibody and/or relative to an anti-LIV1 IgG3 antibody.

The term “antibody-dependent cellular phagocytosis” or “ADCP” refers to the process by which antibody-coated cells are internalized, either in whole or in part, by phagocytic immune cells (e.g., by macrophages, neutrophils and/or dendritic cells) that bind to an Fc region of Ig. In certain exemplary embodiments, an anti-LIV1 IgG1 antibody of the invention mediates equal or increased ADCP relative to a parental antibody and/or relative to an anti-LIV1 IgG3 antibody.

The term “complement-dependent cytotoxicity” or “CDC” refers to a mechanism for inducing cell death in which an Fc region of a target-bound antibody activates a series of enzymatic reactions culminating in the formation of holes in the target cell membrane.

Typically, antigen-antibody complexes such as those on antibody-coated target cells bind and activate complement component C1q, which in turn activates the complement cascade leading to target cell death. Activation of complement may also result in deposition of complement components on the target cell surface that facilitate ADCC by binding complement receptors (e.g., CR3) on leukocytes.

A “cytotoxic effect” refers to the depletion, elimination and/or killing of a target cell. A “cytotoxic agent” refers to a compound that has a cytotoxic effect on a cell, thereby mediating depletion, elimination and/or killing of a target cell. In certain embodiments, a cytotoxic agent is conjugated to an antibody or administered in combination with an antibody. Suitable cytotoxic agents are described further herein.

A “cytostatic effect” refers to the inhibition of cell proliferation. A “cytostatic agent” refers to a compound that has a cytostatic effect on a cell, thereby mediating inhibition of growth and/or expansion of a specific cell type and/or subset of cells. Suitable cytostatic agents are described further herein.

The term “patient” or “subject” includes human and other mammalian subjects such as non-human primates, rabbits, rats, mice, and the like and transgenic species thereof, that receive either prophylactic or therapeutic treatment.

The term “effective amount,” in the context of treatment of a LIV1-expressing disorder by administration of an anti-LIV1 antibody or antigen-binding fragment thereof (e.g., a LIV1-ADC) as described herein, refers to an amount of such antibody or antigen-binding fragment thereof that is sufficient to inhibit the occurrence or ameliorate one or more symptoms of a LIV1-related disorder (e.g., a LIV1-expressing cancer). An effective amount of an antibody is administered in an “effective regimen.” The term “effective regimen” refers to a combination of amount of the antibody being administered and dosage frequency adequate to accomplish prophylactic or therapeutic treatment of the disorder (e.g., prophylactic or therapeutic treatment of a LIV1-expressing cancer).

The term “pharmaceutically acceptable” means approved or approvable by a regulatory agency of the Federal or a state government or listed in the U.S. Pharmacopeia or other generally recognized pharmacopeia for use in animals, and more particularly in humans. The term “pharmaceutically compatible ingredient” refers to a pharmaceutically acceptable diluent, adjuvant, excipient, or vehicle with which an anti-LIV1 antibody (e.g., a LIV1-ADC) is formulated.

The phrase “pharmaceutically acceptable salt,” refers to pharmaceutically acceptable organic or inorganic salts. Exemplary salts include sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p toluenesulfonate, and pamoate (i.e., 1,1′-methylene bis-(2 hydroxy-3-naphthoate) salts. A pharmaceutically acceptable salt may further comprise an additional molecule such as, e.g., an acetate ion, a succinate ion or other counterion. A counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counterion.

A “platinum-based therapy” refers to treatment with a platinum-based agent. A “platinum-based agent” refers to a molecule or a composition comprising a molecule containing a coordination complex comprising the chemical element platinum and useful as a chemotherapy drug. Platinum-based agents generally act by inhibiting DNA synthesis and some have alkylating activity. Platinum-based agents encompass those that are currently being used as part of a chemotherapy regimen, those that are currently in development, and those that may be developed in the future.

Unless otherwise apparent from the context, when a value is expressed as “about” X or “approximately” X, the stated value of X will be understood to be accurate to ±10%.

Solvates in the context of the invention are those forms of the compounds of the invention that form a complex in the solid or liquid state through coordination with solvent molecules. Hydrates are one specific form of solvates, in which the coordination takes place with water. In certain exemplary embodiments, solvates in the context of the present invention are hydrates.

### II. ANTI-LIV1 ANTIBODIES, ANTIGEN-BINDING FRAGMENTS AND ANTIBODY-DRUG CONJUGATES

The present invention provides isolated, recombinant and/or synthetic human, primate, rodent, mammalian, chimeric, humanized and/or CDR-grafted antibodies and antigen-binding fragments and antibody-drug conjugates (e.g., a LIV1-ADC) thereof, as well as compositions and nucleic acid molecules comprising at least one polynucleotide encoding at least a portion of one antibody molecule. The present invention further includes, but is not limited to, methods of making and using such nucleic acids and antibodies including diagnostic and therapeutic compositions, methods and devices. In certain exemplary embodiments, humanized anti-LIV1 IgG1 antibodies are provided. In other exemplary embodiments, humanized anti-LIV1 IgG1 antibody-drug conjugates are provided.

In some embodiments, the invention provides an antibody-drug conjugate for the treatment of cancer. In some embodiments, the antibody-drug conjugate comprises an antibody conjugated to an auristatin. In some embodiments, the auristatin is a monomethyl auristatin. In some embodiments, the monomethyl auristatin is monomethyl auristatin E.

Unless otherwise indicated, an anti-LIV1-antibody drug conjugate (i.e., a LIV1-ADC) includes an antibody specific for the human LIV1 protein conjugated to a cytotoxic agent. SGN-LIV1A is an anti-LIV1 humanized antibody (also referred to as hLIV22) which is conjugated to monomethyl auristatin E (MMAE) via a protease-cleavable linker (i.e., a valine-citrulline linker). Upon binding to a LIV1 expressing cell, SGN-LIV1A is internalized and releases MMAE, which disrupts microtubulin and induces apoptosis. SGN-LIV1A is also known as ladiratuzumab vedotin.

SGN-LIV1A comprises a humanized form of the mouse BR2-22a antibody, described in U.S. Pat. No. 9,228,026. Methods of making the SGN-LIV1A antibody are also disclosed in U.S. Pat. No. 9,228,026, which is incorporated herein by reference in its entirety for all purposes.

The amino acid sequence of the heavy chain variable region of SGN-LIV1A is provided herein as SEQ ID NO: 1. The amino acid sequence of the light chain variable region of SGN-LIV1A is provided herein as SEQ ID NO: 2. Synthesis and conjugation of the drug linker vcMMAE (shown below; also referred to as 1006) are further described in U.S. Pat. No. 9,228,026 and US Patent Pub. No. 2005/0238649, which are incorporated herein by reference in their entireties for all purposes.

TABLE 1
HCVR of SGN-LIV1A (SEQ ID NO: 1).

Gln Val Gln Leu Val Gln Ser Gly Ala Glu Val Lys
Lys Pro Gly Ala Ser Val Lys Val Ser Cys Lys Ala
Ser Gly Leu Thr Ile Glu Asp Tyr Tyr Met His Trp
Val Arg Gln Ala Pro Gly Gln Gly Leu Glu Trp Met
Gly Trp Ile Asp Pro Glu Asn Gly Asp Thr Glu Tyr
Gly Pro Lys Phe Gln Gly Arg Val Thr Met Thr Arg
Asp Thr Ser Ile Asn Thr Ala Tyr Met Glu Leu Ser
Arg Leu Arg Ser Asp Asp Thr Ala Val Tyr Tyr Cys
Ala Val His Asn Ala His Tyr Gly Thr Trp Phe Ala
Tyr Trp Gly Gln Gly Thr Leu Val Thr Val Ser Ser

TABLE 2
LCVR of SGN-LIV1A (SEQ ID NO: 2).

Asp Val Val Met Thr Gln Ser Pro Leu Ser Leu Pro
Val Thr Leu Gly Gln Pro Ala Ser Ile Ser Cys Arg
Ser Ser Gln Ser Leu Leu His Ser Ser Gly Asn Thr
Tyr Leu Glu Trp Tyr Gln Gln Arg Pro Gly Gln Ser
Pro Arg Pro Leu Ile Tyr Lys Ile Ser Thr Arg Phe
Ser Gly Val Pro Asp Arg Phe Ser Gly Ser Gly Ser
Gly Thr Asp Phe Thr Leu Lys Ile Ser Arg Val Glu
Ala Glu Asp Val Gly Val Tyr Tyr Cys Phe Gln Gly
Ser His Val Pro Tyr Thr Phe Gly Gly Gly Thr Lys
Val Glu Ile Lys Arg

According to certain exemplary embodiments, a LIV1-ADC comprises monomethyl auristatin E (MMAE) (PubChem CID: 53297465):

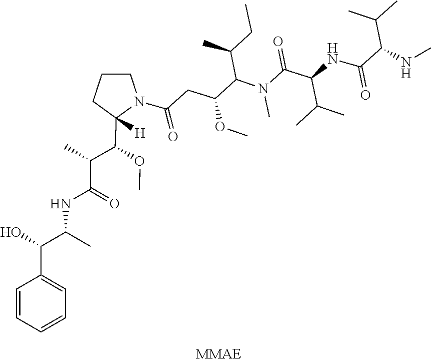 [SMILES: CC[C@H](C)C([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](NC)C(C)C)C(C)C]

According to certain exemplary embodiments, a LIV1-ADC comprises vcMMAE conjugated thereto. vcMMAE is a drug-linker conjugate for ADC with potent anti-tumor activity comprising the anti-mitotic agent, MMAE, linked via the lysosomally cleavable dipeptide valine-citrulline (vc):

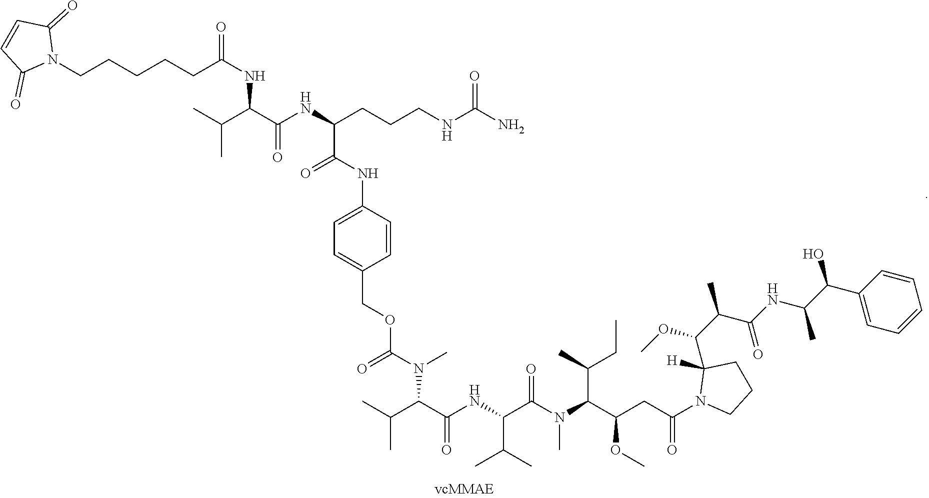 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](NC(=O)CCCCCN2C(=O)C=CC2=O)C(C)C)cc1)C(C)C]

U.S. Pat. No. 9,228,026 discloses methods for conjugating vcMMAE to hLIV22.

A vcMMAE-antibody conjugate (e.g., a LIV1-ADC) according to certain exemplary embodiments is set forth below.

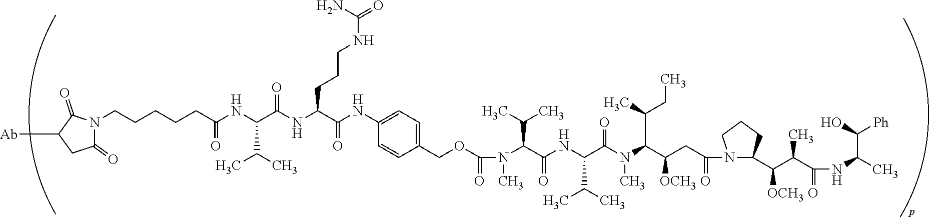

According to certain exemplary embodiments, a vcMMAE-antibody conjugate (e.g., a LIV1-ADC) is provided as set forth above, wherein Ab may include an anti-LIV1 antibody or antigen-binding fragment thereof (e.g., hLIV22), and wherein p may be any integer from about 1 to about 8. In some embodiments, a vcMMAE-antibody conjugate (e.g., a LIV1-ADC) is provided as set forth above, wherein Ab may include an anti-LIV1 antibody or antigen-binding fragment thereof (e.g., hLIV22), and wherein p is 1, representing a vcMMAE to antibody or antigen-binding fragment thereof ratio of 1. In some embodiments, a vcMMAE-antibody conjugate (e.g., a LIV1-ADC) is provided as set forth above, wherein Ab may include an anti-LIV1 antibody or antigen-binding fragment thereof (e.g., hLIV22), and wherein p is 2, 3, 4, 5, 6, 7, 8, 9, or 10, representing a vcMMAE to antibody or antigen-binding fragment thereof ratio (also known as a “Drug-to-Antibody Ratio” or “DAR”) of 2, 3, 4, 5, 6, 7, 8, 9, or 10, respectively. Accordingly, in some embodiments, a vcMMAE-antibody conjugate (e.g., a LIV1-ADC) is provided as set forth above, wherein a vcMMAE to antibody or antigen-binding fragment thereof ratio is 1, 2, 3, 4, 5, 6, 7, 8, 9, or 10. In certain exemplary embodiments, a vcMMAE-antibody conjugate (e.g., a LIV1-ADC) is provided as set forth above, wherein Ab may include an anti-LIV1 antibody or antigen-binding fragment thereof (e.g., hLIV22), and wherein p is 4, representing a vcMMAE to antibody or antigen-binding fragment thereof ratio of 4. Accordingly, in certain exemplary embodiments, a vcMMAE-antibody conjugate (e.g., a LIV1-ADC) is provided as set forth above, wherein a vcMMAE to antibody or antigen-binding fragment thereof ratio is 4.

SGN-LIV1A can be administered to subjects at a level that inhibits cancer cell growth, while at the same time is tolerated by the subject.

In certain exemplary embodiments, an anti-LIV1 antibody or antigen-binding fragment thereof comprises CDRs from an HCVR set forth as SEQ ID NO: 1 and/or CDRs from an LCVR set forth as SEQ ID NO: 2. In certain exemplary embodiments, an anti-LIV1 antibody or antigen-binding fragment thereof comprises an HCVR set forth as SEQ ID NO: 1 and/or an LCVR set forth as SEQ ID NO: 2. In other embodiments, an anti-LIV1 antibody or antigen-binding fragment thereof comprises an HCVR/LCVR pair SEQ ID NO: 1/SEQ ID NO: 2. In other embodiments, an anti-LIV1 antibody or antigen-binding fragment thereof comprises an HCVR that has at least about 80% homology or identity (e.g., 80%, 85%, 90%, 91%, 92%, 93%, 94%, 95%, 96%, 97%, 98% or 99%) to SEQ ID NO: 1 and/or comprises an LCVR that has at least about 80% homology or identity (e.g., 80%, 85%, 90%, 91%, 92%, 93%, 94%, 95%, 96%, 97%, 98% or 99%) to SEQ ID NO: 2.

Antibodies and antigen-binding fragments thereof and antibody-drug conjugates described herein (e.g., anti-LIV1 antibodies or LIV1-ADCs) can be expressed in a modified form. For instance, a region of additional amino acids, particularly charged amino acids, can be added to the N-terminus of an antibody or an antigen-binding fragment thereof or antibody-drug conjugates (e.g., a LIV1-ADC) to improve stability and persistence in the host cell, during purification, or during subsequent handling and storage. Also, peptide moieties can be added to an antibody or an antigen-binding fragment thereof or antibody-drug conjugates (e.g., a LIV1-ADC) of the present invention to facilitate purification. Such regions can be removed prior to final preparation of an antibody molecule or at least one fragment thereof. Such methods are described in many standard laboratory manuals, such as Sambrook, supra; Ausubel, et al., ed., Current Protocols In Molecular Biology, John Wiley & Sons, Inc., NY, N.Y. (1987-2001).

The antibodies or antigen-binding fragments thereof or antibody-drug conjugates (e.g., anti-LIV1 antibodies or LIV1-ADCs) described herein typically bind the target antigen (e.g., LIV1) with an equilibrium binding constant of about ≤1 μM, e.g., about 100 nM, about 10 nM, or about 1 nM, as measured using standard binding assays, for example, a Biacore-based binding assay.

Antibody molecules of the present invention may be characterized relative to a reference anti-LIV1 antibody, for example, BR2-22a. Antibody BR2-22a is described in U.S. Pat. No. 8,591,863 and is commercially available from American Type Culture Collection.

### Antibody-Drug Conjugates

In certain embodiments, the antibodies of the invention (e.g., anti-LIV1 antibodies) can be conjugated to a drug to form antibody-drug conjugates (ADCs). An exemplary anti-LIV1-ADC antibody is SGN-LIV1A. Particular ADCs may comprise cytotoxic agents (e.g., chemotherapeutic agents), prodrug converting enzymes, radioactive isotopes or compounds, or toxins (these moieties being collectively referred to as a therapeutic agent). For example, an ADC can be conjugated to a cytotoxic agent such as a chemotherapeutic agent, or a toxin (e.g., a cytostatic or cytocidal agent such as, for example, abrin, ricin A, pseudomonas exotoxin, or diphtheria toxin). Examples of useful classes of cytotoxic agents include, for example, DNA minor groove binders, DNA replication inhibitors, chemotherapy sensitizers, DNA alkylating agents, and tubulin inhibitors. Other exemplary classes of cytotoxic agents include anthracyclines, auristatins, camptothecins, duocarmycins, etoposides, maytansinoids and vinca alkaloids. Some exemplary cytotoxic agents include auristatins (e.g., auristatin T, auristatin E, AFP, monomethyl auristatin F (MMAF), lipophilic monomethyl aurstatin F, monomethyl auristatin E (MMAE)), DNA minor groove binders (e.g., enediynes and lexitropsins), duocarmycins, taxanes (e.g., paclitaxel and docetaxel), vinca alkaloids, nicotinamide phosphoribosyltranferase inhibitor (NAMPTi), tubulysin M, doxorubicin, morpholino-doxorubicin, and cyanomorpholino-doxorubicin.

The cytotoxic agent can be a chemotherapeutic such as, for example, doxorubicin, paclitaxel, melphalan, vinca alkaloids, methotrexate, mitomycin C or etoposide. The agent can also be a CC-1065 analogue, calicheamicin, maytansine, an analog of dolastatin 10, rhizoxin, or palytoxin.

The cytotoxic agent can also be an auristatin. The auristatin can be an auristatin E derivative is, e.g., an ester formed between auristatin E and a keto acid. For example, auristatin E can be reacted with paraacetyl benzoic acid or benzoylvaleric acid to produce AEB and AEVB, respectively. Other typical auristatins include auristatin T, AFP, MMAF, and MMAE. The synthesis and structure of various auristatins are described in, for example, US 2005-0238649 and US2006-0074008.

The cytotoxic agent can be a DNA minor groove binding agent. (See, e.g., U.S. Pat. No. 6,130,237.) For example, the minor groove binding agent can be a CBI compound or an enediyne (e.g., calicheamicin).

The cytotoxic or cytostatic agent can be an anti-tubulin agent. Examples of anti-tubulin agents include taxanes (e.g., Taxol® (paclitaxel), Taxotere® (docetaxel)), T67 (Tularik), vinca alkyloids (e.g., vincristine, vinblastine, vindesine, and vinorelbine), and auristatins (e.g., auristatin E, AFP, MMAF, MMAE, AEB, AEVB). Other suitable antitubulin agents include, for example, baccatin derivatives, taxane analogs (e.g., epothilone A and B), nocodazole, colchicine and colcimid, estramustine, cryptophysins, cemadotin, maytansinoids, combretastatins, discodermoide and eleuthrobin.

The cytotoxic agent can be a maytansinoid, another group of anti-tubulin agents (e.g., DM1, DM2, DM3, DM4). For example, the maytansinoid can be maytansine or a maytansine containing drug linker such as DM-1 or DM-4 (ImmunoGen, Inc.; see also Chari et al., 1992, Cancer Res.).

An ADC can be conjugated to a pro-drug converting enzyme. The pro-drug converting enzyme can be recombinantly fused to the antibody or chemically conjugated thereto using known methods. Exemplary pro-drug converting enzymes are carboxypeptidase G2, beta-glucuronidase, penicillin-V-amidase, penicillin-G-amidase, β-lactamase, β-glucosidase, nitroreductase and carboxypeptidase A.

Techniques for conjugating therapeutic agents to proteins, and in particular to antibodies, are well-known. (See, e.g., Alley et al., Current Opinion in Chemical Biology 2010 14: 1-9; Senter, Cancer J., 2008, 14(3): 154-169.) The therapeutic agent can be conjugated in a manner that reduces its activity unless it is cleaved off the antibody (e.g., by hydrolysis, by proteolytic degradation, or by a cleaving agent). In some aspects, the therapeutic agent is attached to the antibody with a cleavable linker that is sensitive to cleavage in the intracellular environment of the LIV1-expressing cancer cell but is not substantially sensitive to the extracellular environment, such that the conjugate is cleaved from the antibody when it is internalized by the LIV1-expressing cancer cell (e.g., in the endosomal or, for example by virtue of pH sensitivity or protease sensitivity, in the lysosomal environment or in the caveolear environment). In some embodiments, the therapeutic agent can also be attached to the antibody with a non-cleavable linker.

In certain exemplary embodiments, an ADC can include a linker region between a cytotoxic or cytostatic agent and the antibody. As noted supra, typically, the linker can be cleavable under intracellular conditions, such that cleavage of the linker releases the therapeutic agent from the antibody in the intracellular environment (e.g., within a lysosome or endosome or caveolea). The linker can be, e.g., a peptidyl linker that is cleaved by an intracellular peptidase or protease enzyme, including a lysosomal or endosomal protease. Cleaving agents can include cathepsins B and D and plasmin (see, e.g., Dubowchik and Walker, Pharm. Therapeutics 83:67-123, 1999). Most typical are peptidyl linkers that are cleavable by enzymes that are present in LIV1-expressing cells. For example, a peptidyl linker that is cleavable by the thiol-dependent protease cathepsin-B, which is highly expressed in cancerous tissue, can be used (e.g., a linker comprising a Phe-Leu or a Val-Cit peptide).

A cleavable linker can be pH-sensitive, i.e., sensitive to hydrolysis, at certain pH values. Typically, a pH-sensitive linker is hydrolyzable under acidic conditions. For example, an acid-labile linker that is hydrolyzable in the lysosome (e.g., a hydrazone, semicarbazone, thiosemicarbazone, cis-aconitic amide, orthoester, acetal, ketal, or the like) can be used. (See, e.g., U.S. Pat. Nos. 5,122,368; 5,824,805; 5,622,929; Dubowchik and Walker, Pharm. Therapeutics 83:67-123, 1999; Neville et al, Biol. Chem. 264: 14653-14661, 1989.) Such linkers are relatively stable under neutral pH conditions, such as those in the blood, but are unstable at below pH 5.5 or 5.0, the approximate pH of the lysosome.

Other linkers are cleavable under reducing conditions (e.g., a disulfide linker). Disulfide linkers include those that can be formed using SATA (N-succinimidyl-S-acetylthioacetate), SPDP (N-succinimidyl-3-(2-pyridyldithio)propionate), SPDB (N-succinimidyl-3-(2-pyridyldithio)butyrate) and SMPT (N-succinimidyl-oxycarbonyl-alpha-methyl-alpha-(2-pyridyl-dithio)toluene), SPDB and SMPT. (See, e.g., Thorpe et al., Cancer Res. 47:5924-5931, 1987; Wawrzynczak et al., In Immunoconjugates: Antibody Conjugates in Radioimagery and Therapy of Cancer (C. W. Vogel ed., Oxford U. Press, 1987. See also U.S. Pat. No. 4,880,935.)

A linker can also be a malonate linker (Johnson et al., Anticancer Res. 15: 1387-93, 1995), a maleimidobenzoyl linker (Lau et al., Bioorg-Med-Chem. 3: 1299-1304, 1995), or a 3′-N-amide analog (Lau et al., Bioorg-Med-Chem. 3: 1305-12, 1995).

A linker also can be a non-cleavable linker, such as a maleimido-alkylene or maleimide-aryl linker that is directly attached to the therapeutic agent and released by proteolytic degradation of the antibody.

Typically, a linker is not substantially sensitive to the extracellular environment, meaning that no more than about 20%, typically no more than about 15%, more typically no more than about 10%, and even more typically no more than about 5%, no more than about 3%, or no more than about 1% of the linkers in a sample of the ADC are cleaved when the ADC is present in an extracellular environment (e.g., in plasma). Whether a linker is not substantially sensitive to the extracellular environment can be determined, for example, by incubating independently with plasma both (a) the ADC (the “ADC sample”) and (b) an equal molar amount of unconjugated antibody or therapeutic agent (the “control sample”) for a predetermined time period (e.g., 2, 4, 8, 16, or 24 hours) and then comparing the amount of unconjugated antibody or therapeutic agent present in the ADC sample with that present in control sample, as measured, for example, by high performance liquid chromatography.

A linker can also promote cellular internalization, e.g., when conjugated to the therapeutic agent (i.e., in the milieu of the linker-therapeutic agent moiety of the ADC or ADC derivate as described herein). Alternatively, the linker can promote cellular internalization when conjugated to both the therapeutic agent and the antibody (i.e., in the milieu of the ADC as described herein).

An antibody (e.g., anti-LIV1 antibody) can be conjugated to a linker via a heteroatom of the antibody. These heteroatoms can be present on the antibody in its natural state or can be introduced into the antibody (e.g., anti-LIV1 antibody). In some aspects, the antibody (e.g., anti-LIV1 antibody) will be conjugated to the linker via a sulfur atom of a cysteine residue. Methods of conjugating linker and drug-linkers to antibodies are known in the art.

Exemplary antibody-drug conjugates include auristatin based antibody-drug conjugates meaning that the drug component is an auristatin drug. Auristatins bind tubulin, have been shown to interfere with microtubule dynamics and nuclear and cellular division, and have anticancer activity. Typically the auristatin based antibody-drug conjugate comprises a linker between the auristatin drug and the antibody (e.g., anti-LIV1 antibody). The linker can be, for example, a cleavable linker (e.g., a peptidyl linker) or a non-cleavable linker (e.g., linker released by degradation of the antibody). Auristatins include MMAF and MMAE. The synthesis and structure of exemplary auristatins are described in U.S. Pat. Nos. 7,659,241, 7,498,298, 7,968,687, and U.S. Pub. Nos. 2009/0111756 and 2009/0018086, each of which is incorporated herein by reference in its entirety and for all purposes.

In certain embodiments, an antibody or antigen-binding fragment thereof can be conjugated to a drug to form an antibody-drug conjugate (ADC) and may have a ratio of drug moieties per antibody of about 1 to about 8. In certain embodiments, an antibody or antigen-binding fragment thereof (e.g., anti-LIV1 antibody) can be conjugated to a drug to form an ADC and may have a ratio of drug moieties per antibody of about 2 to about 5. In some embodiments, the ratio of drug moieties per antibody is 1, 2, 3, 4, 5, 6, 7, 8, 9, or 10. In certain exemplary embodiments, an anti-LIV1 antibody or antigen-binding fragment thereof can be conjugated to a drug to form an ADC and have a ratio of drug moieties per antibody of about 4. In some embodiments, the average number of drug moieties per antibody in a population of antibody-drug conjugates is about 1 to about 8. In some embodiments, the average number of drug moieties per antibody in a population of antibody-drug conjugates is about 4. Methods of determining the ratio of drug moieties per antibody or antigen-binding fragment thereof of an ADC are readily known to those skilled in the art.

### III. THERAPEUTIC APPLICATIONS

The invention provides methods of treating cancer. In cancer cells express LIV1. In one aspect, the invention provides the use of humanized anti-LIV1 antibodies and antigen-binding fragments or conjugates thereof (e.g., anti-LIV1-antibody-drug conjugates (anti-LIV1-ADCs)) for the treatment of cancers, such as solid tumors, such as, e.g., locally advanced or metastatic solid tumors (e.g., prostate cancer and melanoma).

As used herein, the terms “subject” and “patient” refer to organisms to be treated by the methods of the present invention. Such organisms preferably include, but are not limited to, mammals (e.g., murines, simians, equines, bovines, porcines, canines, felines, and the like), and more preferably includes humans. As used herein, the terms “treat,” “treatment” and “treating” include any effect, e.g., lessening, reducing, modulating, ameliorating or eliminating, that results in the improvement of the condition, disease, disorder, and the like, or ameliorating a symptom thereof, such as for example, reduced number of cancer cells, reduced tumor size, reduced rate of cancer cell infiltration into peripheral organs, or reduced rate of tumor metastasis or tumor growth.

Positive therapeutic effects in cancer can be measured in a number of ways (See, W. A. Weber, J. Null. Med. 50:1S-10S (2009); Eisenhauer et al., supra). In certain exemplary embodiments, response to an anti-LIV1 antibody or an antigen-binding fragment thereof (e.g., a LIV1-ADC) is assessed using RECIST 1.1 criteria. In some embodiments, the treatment achieved by a therapeutically effective amount is any of a partial response (PR), a complete response (CR), progression free survival (PFS), disease free survival (DFS), objective response (OR) or overall survival (OS). The dosage regimen of a therapy described herein that is effective to treat a cancer patient may vary according to factors such as the disease state, age, and weight of the patient, and the ability of the therapy to elicit an anti-cancer response in the subject. While an embodiment of the treatment method, medicaments and uses of the present invention may not be effective in achieving a positive therapeutic effect in every subject, it should do so in a statistically significant number of subjects as determined by any statistical test known in the art such as the Student's t-test, the chi²-test, the U-test according to Mann and Whitney, the Kruskal-Wallis test (H-test), Jonckheere-Terpstra-test and the Wilcoxon-test.

“RECIST 1.1 Response Criteria” as used herein means the definitions set forth in Eisenhauer et al., E. A. et al., Eur. J Cancer 45:228-247 (2009) for target lesions or non-target lesions, as appropriate, based on the context in which response is being measured.

“Tumor” as it applies to a subject diagnosed with, or suspected of having, cancer (e.g., prostate cancer or melanoma), refers to a malignant or potentially malignant neoplasm or tissue mass of any size.

“Tumor burden” also referred to as “tumor load,” refers to the total amount of tumor material distributed throughout the body. Tumor burden refers to the total number of cancer cells or the total size of tumor(s) throughout the body, including lymph nodes and bone narrow. Tumor burden can be determined by a variety of methods known in the art, such as, e.g., by measuring the dimensions of tumor(s) upon removal from the subject, e.g., using calipers, or while in the body using imaging techniques, e.g., ultrasound, bone scan, computed tomography (CT) or magnetic resonance imaging (MRI) scans.

The term “tumor size” refers to the total size of the tumor which can be measured as the length and width of a tumor. Tumor size may be determined by a variety of methods known in the art, such as, e.g. by measuring the dimensions of tumor(s) upon removal from the subject, e.g., using calipers, or while in the body using imaging techniques, e.g., bone scan, ultrasound, CT or MRI scans.

As used herein, the term “effective amount” refers to the amount of a compound (e.g., an anti-LIV1 antibody or antigen-binding fragment thereof or antibody-drug conjugate) sufficient to effect beneficial or desired results. An effective amount of an antibody or antigen-binding fragment thereof or antibody-drug conjugate (e.g., a LIV1-ADC) can be administered in one or more administrations, applications or dosages and is not intended to be limited to a particular formulation or administration route. Generally, a therapeutically effective amount of an antibody or antigen-binding fragment thereof or antibody-drug conjugate (e.g., a LIV1-ADC) is in the range of 0.2 mg/kg to 3.5 mg/kg of body weight of the subject. In some embodiments, the maximum dose is about 100 mg, about 125 mg, about 200 mg or about 250 mg. In some embodiments, a therapeutically effective amount of an antibody or antigen-binding fragment thereof or antibody-drug conjugate (e.g., a LIV1-ADC) is in the range of 0.5 mg/kg to 2.8 mg/kg at a maximum dose of about 100 mg, about 125 mg, about 200 mg, or about 250 mg. In some embodiments, a therapeutically effective amount of an antibody or antigen-binding fragment thereof or antibody-drug conjugate (e.g., a LIV1-ADC) is in the range of 0.5 mg/kg to 3.0 mg/kg of body weight of the subject at a maximum dose of about 200 mg. In some embodiments, a therapeutically effective amount of an antibody or antigen-binding fragment thereof or antibody-drug conjugate (e.g., a LIV1-ADC) is in the range of 0.5 mg/kg to 2.0 mg/kg of body weight of the subject. In some embodiments, a therapeutically effective amount of an antibody or antigen-binding fragment thereof or antibody-drug conjugate (e.g., a LIV1-ADC) is in the range of 0.75 mg/kg to 1.67 mg/kg of body weight of the subject. In some embodiments, a therapeutically effective amount of an antibody or antigen-binding fragment thereof or antibody-drug conjugate (e.g., a LIV1-ADC) is in the range of 0.5 mg/kg to 3.0 mg/kg of body weight of the subject. In some embodiments, a therapeutically effective amount of an antibody or antigen-binding fragment thereof or antibody-drug conjugate (e.g., a LIV1-ADC) is in the range of 1.0 mg/kg to 2.5 mg/kg of body weight of the subject. In some embodiments, a therapeutically effective amount of an antibody or antigen-binding fragment thereof or antibody-drug conjugate (e.g., a LIV1-ADC) is about 0.75 mg/kg of body weight of the subject. In some embodiments, a therapeutically effective amount of an antibody or antigen-binding fragment thereof or antibody-drug conjugate (e.g., a LIV1-ADC) is 0.75 mg/kg of body weight of the subject. In some embodiments, a therapeutically effective amount of an antibody or antigen-binding fragment thereof or antibody-drug conjugate (e.g., a LIV1-ADC) is about 1.0 mg/kg of body weight of the subject. In some embodiments, a therapeutically effective amount of an antibody or antigen-binding fragment thereof or antibody-drug conjugate (e.g., a LIV1-ADC) is 1.0 mg/kg of body weight of the subject. In some embodiments, a therapeutically effective amount of an antibody or antigen-binding fragment thereof or antibody-drug conjugate (e.g., a LIV1-ADC) is about 1.25 mg/kg of body weight of the subject. In some embodiments, a therapeutically effective amount of an antibody or antigen-binding fragment thereof or antibody-drug conjugate (e.g., a LIV1-ADC) is 1.25 mg/kg of body weight of the subject. In some embodiments, a therapeutically effective amount of an antibody or antigen-binding fragment thereof or antibody-drug conjugate (e.g., a LIV1-ADC) is about 1.5 mg/kg of body weight of the subject. In some embodiments, a therapeutically effective amount of an antibody or antigen-binding fragment thereof or antibody-drug conjugate (e.g., a LIV1-ADC) is 1.5 mg/kg of body weight of the subject. In some embodiments, a therapeutically effective amount of an antibody or antigen-binding fragment thereof or antibody-drug conjugate (e.g., a LIV1-ADC) is about 1.67 mg/kg of body weight of the subject. In some embodiments, a therapeutically effective amount of an antibody or antigen-binding fragment thereof or antibody-drug conjugate (e.g., a LIV1-ADC) is 1.67 mg/kg of body weight of the subject. In some embodiments, a therapeutically effective amount of an antibody or antigen-binding fragment thereof or antibody-drug conjugate (e.g., a LIV1-ADC) is about 1.75 mg/kg of body weight of the subject. In some embodiments, a therapeutically effective amount of an antibody or antigen-binding fragment thereof or antibody-drug conjugate (e.g., a LIV1-ADC) is 1.75 mg/kg of body weight of the subject. In some embodiments, a therapeutically effective amount of an antibody or antigen-binding fragment thereof or antibody-drug conjugate (e.g., a LIV1-ADC) is about 2.0 mg/kg of body weight of the subject. In some embodiments, a therapeutically effective amount of an antibody or antigen-binding fragment thereof or antibody-drug conjugate (e.g., a LIV1-ADC) is 2.0 mg/kg of body weight of the subject. In some embodiments, a therapeutically effective amount of an antibody or antigen-binding fragment thereof or antibody-drug conjugate (e.g., a LIV1-ADC) is about 2.25 mg/kg of body weight of the subject. In some embodiments, a therapeutically effective amount of an antibody or antigen-binding fragment thereof or antibody-drug conjugate (e.g., a LIV1-ADC) is 2.5 mg/kg of body weight of the subject. The dosage administered can vary depending upon known factors, such as the pharmacodynamic characteristics of the particular agent, and its mode and route of administration; the age, health, and weight of the recipient; the type and extent of disease or indication to be treated, the nature and extent of symptoms, kind of concurrent treatment, frequency of treatment, and the effect desired. The initial dosage can be increased beyond the upper level in order to rapidly achieve the desired blood-level or tissue-level. Alternatively, the initial dosage can be smaller than the optimum, and the daily dosage may be progressively increased during the course of treatment. Dosing frequency can vary, depending on factors such as route of administration, dosage amount, serum half-life of the antibody, and the disease being treated. Exemplary dosing frequencies are once per day, once per week, once every two weeks and once every three weeks. Formulation of monoclonal antibody-based drugs is within ordinary skill in the art. In some embodiments, a monoclonal antibody is lyophilized, and then reconstituted in buffered saline, at the time of administration.

In some embodiments, an antibody or antigen-binding fragment thereof or antibody-drug conjugate (e.g., a LIV1-ADC) is administered to a patient who failed to achieve a sustained response after prior therapy (e.g., after failed or ineffective therapy with a systemic anti-cancer therapy that is not the antibody or antigen-binding fragment thereof or antibody-drug conjugate (e.g., a LIV1-ADC)), i.e., is cancer treatment-experienced.

In some embodiments, a medicament comprising an antibody or antigen-binding fragment thereof or antibody-drug conjugate (e.g., a LIV1-ADC), as described above, may be provided as a liquid formulation or prepared by reconstituting a lyophilized powder with sterile water for injection prior to use.

In certain embodiments, the dosing regimen will comprise administering an anti-LIV1 antibody or antigen-binding fragment thereof or antibody-drug conjugate (e.g., a LIV1-ADC) once about every week, once about every two weeks, once about every three weeks or once about every month.

In certain embodiments, the dosing regimen will comprise administering an anti-LIV1 antibody or antigen-binding fragment thereof or antibody-drug conjugate (e.g., a LIV1-ADC) at a dose of about 2.5 mg/kg of a subject's body weight at intervals of about 21 days (±2 days) throughout the course of treatment. In certain embodiments, an anti-LIV1 antibody or antigen-binding fragment thereof (e.g., a LIV1-ADC) is used at a dose of less than about 200 mg every 3 weeks.

In certain embodiments, the dosing regimen will comprise administering an anti-LIV1 antibody or antigen-binding fragment thereof or antibody-drug conjugate (e.g., a LIV1-ADC) at a dose of about 1.0 mg/kg of a subject's body weight at intervals of about 7 days (±1 day) throughout the course of treatment. In certain embodiments, an anti-LIV1 antibody or antigen-binding fragment thereof (e.g., a LIV1-ADC) is used at a dose of less than about 100 mg every 1 week. In certain embodiments, an anti-LIV1 antibody or antigen-binding fragment thereof (e.g., a LIV1-ADC) is used at a dose of less than about 200 mg every 1 week.

In certain embodiments, the dosing regimen will comprise administering an anti-LIV1 antibody or antigen-binding fragment thereof or antibody-drug conjugate (e.g., a LIV1-ADC) at a dose of about 1.25 mg/kg of a subject's body weight at intervals of about 7 days (±1 day) throughout the course of treatment. In certain embodiments, an anti-LIV1 antibody or antigen-binding fragment thereof (e.g., a LIV1-ADC) is used at a dose of less than about 125 mg every 1 week. In certain embodiments, an anti-LIV1 antibody or antigen-binding fragment thereof (e.g., a LIV1-ADC) is used at a dose of less than about 200 mg every 1 week.

In certain embodiments, the dosing regimen will comprise administering an anti-LIV1 antibody or antigen-binding fragment thereof or antibody-drug conjugate (e.g., a LIV1-ADC) at a dose of about 2.5 mg/kg of a subject's body weight at intervals of about 21 days (±2 days) throughout the course of treatment. In certain embodiments, an anti-LIV1 antibody or antigen-binding fragment thereof or antibody-drug conjugate (e.g., a LIV1-ADC) is used at a dose of less than or equal to about 250 mg every 3 weeks. In certain embodiments, an anti-LIV1 antibody or antigen-binding fragment thereof or antibody-drug conjugate (e.g., a LIV1-ADC) is used at a dose of less than or equal to 250 mg every 3 weeks. In certain embodiments, the subject is further administered granulocyte colony stimulating factor (G-CSF). In certain embodiments, if the anti-LIV1 antibody or antigen-binding fragment thereof or antibody-drug conjugate (e.g., a LIV1-ADC) is used at a dose of greater than or equal to about 200 mg and less than or equal to about 250 mg every 3 weeks, the subject is further administered G-CSF. In certain embodiments, if the anti-LIV1 antibody or antigen-binding fragment thereof or antibody-drug conjugate (e.g., a LIV1-ADC) is used at a dose of greater than or equal to 200 mg and less than or equal to 250 mg every 3 weeks, the subject is further administered G-CSF. In certain embodiments, the G-CSF is administered prophylactically. In certain embodiments, the G-CSF is recombinant human G-CSF. In certain embodiments, the G-CSF is filgrastim (NEUPOGEN®). In certain embodiments, the G-CSF is PEG-filgrastim (NEULASTA®). In certain embodiments, the G-CSF is lenograstim (GRANOCYTE®). In certain embodiments, the G-CSF is tbo-filgrastim (GRANIX®).

In certain embodiments, the dosing regimen will comprise administering an anti-LIV1 antibody or antigen-binding fragment thereof or antibody-drug conjugate (e.g., a LIV1-ADC) at a dose of about 1.0 mg/kg of a subject's body weight at intervals of about 7 days (±1 day) throughout the course of treatment. In certain embodiments, an anti-LIV1 antibody or antigen-binding fragment thereof or antibody-drug conjugate (e.g., a LIV1-ADC) is used at a dose of less than or equal to about 200 mg every 1 week. In certain embodiments, an anti-LIV1 antibody or antigen-binding fragment thereof or antibody-drug conjugate (e.g., a LIV1-ADC) is used at a dose of less than or equal to 200 mg every 1 week. In certain embodiments, an anti-LIV1 antibody or antigen-binding fragment thereof or antibody-drug conjugate (e.g., a LIV1-ADC) is used at a dose of less than or equal to about 100 mg every 1 week. In certain embodiments, an anti-LIV1 antibody or antigen-binding fragment thereof or antibody-drug conjugate (e.g., a LIV1-ADC) is used at a dose of less than or equal to 100 mg every 1 week. In certain embodiments, the subject is further administered granulocyte colony stimulating factor (G-CSF). In certain embodiments, if the anti-LIV1 antibody or antigen-binding fragment thereof or antibody-drug conjugate (e.g., a LIV1-ADC) is used at a dose of greater than or equal to about 80 mg and less than or equal to about 100 mg every 1 week, the subject is further administered G-CSF. In certain embodiments, if the anti-LIV1 antibody or antigen-binding fragment thereof or antibody-drug conjugate (e.g., a LIV1-ADC) is used at a dose of greater than or equal to 80 mg and less than or equal to 100 mg every 1 week, the subject is further administered G-CSF. In certain embodiments, the G-CSF is administered prophylactically. In certain embodiments, the G-CSF is recombinant human G-CSF. In certain embodiments, the G-CSF is filgrastim (NEUPOGEN®). In certain embodiments, the G-CSF is PEG-filgrastim (NEULASTA®). In certain embodiments, the G-CSF is lenograstim (GRANOCYTE®). In certain embodiments, the G-CSF is tbo-filgrastim (GRANIX®).

In certain embodiments, the dosing regimen will comprise administering an anti-LIV1 antibody or antigen-binding fragment thereof or antibody-drug conjugate (e.g., a LIV1-ADC) at a dose of about 1.25 mg/kg of a subject's body weight at intervals of about 7 days (±1 day) throughout the course of treatment. In certain embodiments, an anti-LIV1 antibody or antigen-binding fragment thereof or antibody-drug conjugate (e.g., a LIV1-ADC) is used at a dose of less than or equal to about 200 mg every 1 week. In certain embodiments, an anti-LIV1 antibody or antigen-binding fragment thereof or antibody-drug conjugate (e.g., a LIV1-ADC) is used at a dose of less than or equal to 200 mg every 1 week. In certain embodiments, an anti-LIV1 antibody or antigen-binding fragment thereof or antibody-drug conjugate (e.g., a LIV1-ADC) is used at a dose of less than or equal to about 125 mg every 1 week. In certain embodiments, an anti-LIV1 antibody or antigen-binding fragment thereof or antibody-drug conjugate (e.g., a LIV1-ADC) is used at a dose of less than or equal to 125 mg every 1 week. In certain embodiments, the subject is further administered granulocyte colony stimulating factor (G-CSF). In certain embodiments, if the anti-LIV1 antibody or antigen-binding fragment thereof or antibody-drug conjugate (e.g., a LIV1-ADC) is used at a dose of greater than or equal to about 100 mg and less than or equal to about 125 mg every 1 week, the subject is further administered G-CSF. In certain embodiments, if the anti-LIV1 antibody or antigen-binding fragment thereof or antibody-drug conjugate (e.g., a LIV1-ADC) is used at a dose of greater than or equal to 100 mg and less than or equal to 125 mg every 1 week, the subject is further administered G-CSF. In certain embodiments, the G-CSF is administered prophylactically. In certain embodiments, the G-CSF is recombinant human G-CSF. In certain embodiments, the G-CSF is filgrastim (NEUPOGEN®). In certain embodiments, the G-CSF is PEG-filgrastim (NEULASTA®). In certain embodiments, the G-CSF is lenograstim (GRANOCYTE®). In certain embodiments, the G-CSF is tbo-filgrastim (GRANIX®).

In certain embodiments, the dosing regimen will comprise administering an antibody or antigen-binding fragment thereof or antibody-drug conjugate (e.g., a LIV1-ADC) once about every week, once about every two weeks, once about every three weeks or once about every month. In certain embodiments, the subject is further administered granulocyte colony-stimulating factor (G-CSF). In certain embodiments, if the dose is greater than or equal to about 200 mg and less than or equal to about 250 mg Q3W or Q21D, the subject is further administered G-CSF. In certain embodiments, the subject is further administered G-CSF. In certain embodiments, if the dose is greater than or equal to 200 mg and less than or equal to 250 mg, the subject is further administered G-CSF. In certain embodiments, the G-CSF is administered prophylactically. In certain embodiments, the G-CSF is recombinant human G-CSF. In certain embodiments, the G-CSF is filgrastim (NEUPOGEN®). In certain embodiments, the G-CSF is PEG-filgrastim (NEULASTA®). In certain embodiments, the G-CSF is lenograstim (GRANOCYTE®). In certain embodiments, the G-CSF is tbo-filgrastim (GRANIX®).

In certain embodiments, the dosing regimen will comprise administering an antibody or antigen-binding fragment thereof or antibody-drug conjugate (e.g., a LIV1-ADC) at a dose of about 0.5 mg/kg to about 2.0 mg/kg of body weight of the subject at intervals of about 7 days (±1 day) throughout the course of treatment.

In a particular embodiment of the invention, the antibody or antigen-binding fragment thereof or antibody-drug conjugate (e.g., a LIV1-ADC) is administered to the subject a dose selected from the group consisting of about 0.5 mg/kg of body weight every week or every 7 days (±1 day), about 0.6 mg/kg of body weight every week or every 7 days (±1 day), about 0.7 mg/kg of body weight every week or every 7 days (±1 day), about 0.75 mg/kg of body weight every week or every 7 days (±1 day), about 0.8 mg/kg of body weight every week or every 7 days (±1 day), about 0.9 mg/kg of body weight every week or every 7 days (±1 day), about 1.0 mg/kg of body weight every week or every 7 days (±1 day), about 1.1 mg/kg of body weight every week or every 7 days (±1 day), about 1.2 mg/kg of body weight every week or every 7 days (±1 day), about 1.25 mg/kg of body weight every week or every 7 days (±1 day), about 1.3 mg/kg of body weight every week or every 7 days (±1 day), about 1.4 mg/kg of body weight every week or every 7 days (±1 day), about 1.5 mg/kg of body weight every week or every 7 days (±1 day), about 1.6 mg/kg of body weight every week or every 7 days (±1 day), about 1.67 mg/kg of body weight every week or every 7 days (±1 day), about 1.7 mg/kg of body weight every week or every 7 days (±1 day), about 1.75 mg/kg of body weight every week or every 7 days (±1 day), about 1.8 mg/kg of body weight every week or every 7 days (±1 day), about 1.9 mg/kg of body weight every week or every 7 days (±1 day), and about 2.0 mg/kg of body weight every week or every 7 days (±1 day), and maximum equivalents of any of these doses, such as, e.g., less than about 200 mg every week or every 7 days (±1 day).

In a particular embodiment of the invention, the antibody or antigen-binding fragment thereof or antibody-drug conjugate (e.g., a LIV1-ADC) is administered to the subject a dose selected from the group consisting of 0.5 mg/kg of body weight every week or every 7 days (±1 day), 0.6 mg/kg of body weight every week or every 7 days (±1 day), 0.7 mg/kg of body weight every week or every 7 days (±1 day), 0.75 mg/kg of body weight every week or every 7 days (±1 day), 0.8 mg/kg of body weight every week or every 7 days (±1 day), 0.9 mg/kg of body weight every week or every 7 days (±1 day), 1.0 mg/kg of body weight every week or every 7 days (±1 day), 1.1 mg/kg of body weight every week or every 7 days (±1 day), 1.2 mg/kg of body weight every week or every 7 days (±1 day), 1.25 mg/kg of body weight every week or every 7 days (±1 day), 1.3 mg/kg of body weight every week or every 7 days (±1 day), 1.4 mg/kg of body weight every week or every 7 days (±1 day), 1.5 mg/kg of body weight every week or every 7 days (±1 day), 1.6 mg/kg of body weight every week or every 7 days (±1 day), 1.67 mg/kg of body weight every week or every 7 days (±1 day), 1.7 mg/kg of body weight every week or every 7 days (±1 day), 1.75 mg/kg of body weight every week or every 7 days (±1 day), 1.8 mg/kg of body weight every week or every 7 days (±1 day), 1.9 mg/kg of body weight every week or every 7 days (±1 day), and 2.0 mg/kg of body weight every week or every 7 days (±1 day), and maximum equivalents of any of these doses, such as, e.g., less than about 200 mg every week or every 7 days (±1 day).

In a particular embodiment of the invention, an anti-LIV1 antibody or antigen-binding fragment thereof or antibody-drug conjugate (e.g., a LIV1-ADC) is administered to a subject in a liquid medicament at a dose selected from the group consisting of about 0.5 mg/kg of body weight every three weeks (Q3W, Q3wk) or every 21 days (Q21D), about 1.0 mg/kg of body weight Q3W or Q21D, about 1.5 mg/kg of body weight Q3W or Q21D, about 2.0 mg/kg of body weight Q3W or Q21D, about 2.5 mg/kg of body weight Q3W or Q21D, about 2.8 mg/kg of body weight Q3W or Q21D, about 3.0 mg/kg of body weight Q3W or Q21D, about 3.2 mg/kg of body weight Q3W or Q21D, or about 3.5 mg/kg of body weight Q3W or Q21D, and maximum equivalents of any of these doses, such as, e.g., less than about 200 mg Q3W or Q21D.

In a particular embodiment of the invention, an anti-LIV1 antibody or antigen-binding fragment thereof or antibody-drug conjugate (e.g., a LIV1-ADC) is administered to a subject in a liquid medicament at a dose selected from the group consisting of about 0.5 mg/kg of body weight every three weeks (Q3W) or every 21 days (Q21D), about 1.0 mg/kg of body weight Q3W or Q21D, about 1.5 mg/kg of body weight Q3W or Q21D, about 2.0 mg/kg of body weight Q3W or Q21D, about 2.5 mg/kg of body weight Q3W or Q21D, about 2.8 mg/kg of body weight Q3W or Q21D, about 3.0 mg/kg of body weight Q3W or Q21D, about 3.2 mg/kg of body weight Q3W or Q21D, or about 3.5 mg/kg of body weight Q3W or Q21D, and maximum equivalents of any of these doses, such as, e.g., less than or equal to about 250 mg Q3W or Q21D. In certain embodiments, the subject is further administered G-CSF. In certain embodiments, if the dose is greater than or equal to about 200 mg and less than or equal to about 250 mg Q3W or Q21D, the subject is further administered G-CSF. In certain embodiments, if the dose is greater than or equal to 200 mg and less than or equal to 250 mg Q3W or Q21D, the subject is further administered G-CSF. In certain embodiments, the G-CSF is administered prophylactically. In certain embodiments, the G-CSF is recombinant human G-CSF. In certain embodiments, the G-CSF is filgrastim (NEUPOGEN®). In certain embodiments, the G-CSF is PEG-filgrastim (NEULASTA®). In certain embodiments, the G-CSF is lenograstim (GRANOCYTE®). In certain embodiments, the G-CSF is tbo-filgrastim (GRANIX®).

In a particular embodiment of the invention, an anti-LIV1 antibody or antigen-binding fragment thereof or antibody-drug conjugate (e.g., a LIV1-ADC) is administered to a subject in a liquid medicament at a dose selected from the group consisting of about 0.25 mg/kg of body weight every one week (Q1W, Q1wk) or every 7 days (Q7D), about 0.50 mg/kg of body weight Q1W or Q7D, about 0.75 mg/kg of body weight Q1W or Q7D, about 1.0 mg/kg of body weight Q1W or Q7D, about 1.25 mg/kg of body weight Q1W or Q7D, about 1.5 mg/kg of body weight Q1W or Q7D, or about 1.75 mg/kg of body weight Q1W or Q7D, and maximum equivalents of any of these doses, such as, e.g., less than about 100 mg Q1W or Q7D or less than about 125 mg Q1W or Q7D or less than about 200 mg Q1W or Q7D.

In a particular embodiment of the invention, an anti-LIV1 antibody or antigen-binding fragment thereof or antibody-drug conjugate (e.g., a LIV1-ADC) is administered to a subject in a liquid medicament at a dose selected from the group consisting of about 0.25 mg/kg of body weight every one week (Q1W) or every 7 days (Q7D), about 0.5 mg/kg of body weight Q1W or Q7D, about 0.75 mg/kg of body weight Q1W or Q7D, about 1.0 mg/kg of body weight Q1W or Q7D, about 1.25 mg/kg of body weight Q1W or Q7D, about 1.5 mg/kg of body weight Q1W or Q7D, or about 1.75 mg/kg of body weight Q1W or Q7D, and maximum equivalents of any of these doses, such as, e.g., less than or equal to about 100 mg Q1W or Q7D or less than about 125 mg Q1W or Q7D or less than about 200 mg Q1W or Q7D. In certain embodiments, the subject is further administered G-CSF. In certain embodiments, if the dose is greater than or equal to about 80 mg and less than or equal to about 100 mg Q1W or Q7D, the subject is further administered G-CSF. In certain embodiments, if the dose is greater than or equal to 80 mg and less than or equal to 100 mg Q1W or Q7D, the subject is further administered G-CSF. In certain embodiments, if the dose is greater than or equal to about 100 mg and less than or equal to about 125 mg Q1W or Q7D, the subject is further administered G-CSF. In certain embodiments, if the dose is greater than or equal to 100 mg and less than or equal to 125 mg Q1W or Q7D, the subject is further administered G-CSF. In certain embodiments, the G-CSF is administered prophylactically. In certain embodiments, the G-CSF is recombinant human G-CSF. In certain embodiments, the G-CSF is filgrastim (NEUPOGEN®). In certain embodiments, the G-CSF is PEG-filgrastim (NEULASTA®). In certain embodiments, the G-CSF is lenograstim (GRANOCYTE®). In certain embodiments, the G-CSF is tbo-filgrastim (GRANIX®).

In some embodiments, an anti-LIV1 antibody or antigen-binding fragment thereof or antibody-drug conjugate (e.g., a LIV1-ADC) is provided in a dosage of about 10 mg, about 20 mg, about 25 mg, about 30 mg, about 40 mg, about 50 mg, about 60 mg, about 70 mg, about 75 mg, about 80 mg, about 90 mg, about 100 mg, about 110 mg, about 120 mg, about 125 mg, about 130 mg, about 140 mg, about 150 mg, about 160 mg, about 170 mg, about 180 mg, about 190 mg, about 191 mg, about 192 mg, about 193 mg, about 194 mg, about 195 mg, about 196 mg, about 197 mg, about 198 mg, about 199 mg or about 200 mg. In certain exemplary embodiments, an anti-LIV1 antibody or antigen-binding fragment thereof or antibody-drug conjugate (e.g., a LIV1-ADC) is provided in a dosage of less than about 200 mg, e.g., at a dosage of about 200 mg, at a dosage of about 199 mg, about 198 mg, about 197 mg, about 196 mg, about 195 mg, about 190 mg, about 185 mg, about 180 mg, about 175 mg, about 170 mg, about 165 mg, about 160 mg, about 155 mg, about 150 mg, about 145 mg, about 140 mg, about 135 mg, about 130 mg, about 125 mg, about 120 mg, about 115 mg, about 110 mg, about 105 mg, about 100 mg, about 90 mg, about 80 mg, about 75 mg, about 60 mg, about 50 mg, about 40 mg, about 30 mg, about 25 mg, about 20 mg, or about 10 mg.

In some embodiments, an anti-LIV1 antibody or antigen-binding fragment thereof or antibody-drug conjugate (e.g., a LIV1-ADC) is provided in a dosage of about 10 mg, about 20 mg, about 25 mg, about 30 mg, about 40 mg, about 50 mg, about 60 mg, about 70 mg, about 75 mg, about 80 mg, about 90 mg, about 100 mg, about 110 mg, about 120 mg, about 125 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 245 mg or about 250 mg. In certain exemplary embodiments, an anti-LIV1 antibody or antigen-binding fragment thereof or antibody-drug conjugate (e.g., a LIV1-ADC) is provided in a dosage of less than or equal to about 250 mg, e.g., at a dosage of about 250 mg, at a dosage of about 245 mg, about 240 mg, about 235 mg, about 230 mg, about 225 mg, about 220 mg, about 215 mg, about 210 mg, about 205 mg, about 200 mg, about 195 mg, about 190 mg, about 185 mg, about 180 mg, about 175 mg, about 170 mg, about 165 mg, about 160 mg, about 155 mg, about 150 mg, about 145 mg, about 140 mg, about 135 mg, about 130 mg, about 125 mg, about 120 mg, about 115 mg, about 110 mg, about 105 mg, about 100 mg, about 90 mg, about 80 mg, about 75 mg, about 60 mg, about 50 mg, about 40 mg, about 30 mg, about 25 mg, about 20 mg, or about 10 mg.

In certain exemplary embodiments, the present invention provides a method for treating cancer in a cell, tissue, organ, animal or patient. In certain exemplary embodiments, the present invention provides a method for treating a solid tumor in a human. In certain exemplary embodiments, the present invention provides a method for treating prostate cancer in a human. In a particular exemplary embodiment, the prostate cancer is locally advance or metastatic. In certain exemplary embodiments, the present invention provides a method for treating melanoma in a human. In a particular exemplary embodiment, the melanoma is locally advance or metastatic.

In certain embodiments, a subject will be administered a parenteral dosing, e.g., an intravenous (IV) infusion, of a medicament comprising an anti-LIV1 antibody or antigen-binding fragment thereof or antibody-drug conjugate (e.g., a LIV1-ADC).

In certain exemplary embodiments, an antibody or antigen-binding fragment thereof or antibody-drug conjugate (e.g., a LIV1-ADC) is administered as a monotherapy. In certain exemplary embodiments, an antibody or antigen-binding fragment thereof or antibody-drug conjugate (e.g., a LIV1-ADC) is administered in combination with trastuzumab. In certain exemplary embodiments, an antibody or antigen-binding fragment thereof or antibody-drug conjugate (e.g., a LIV1-ADC) is administered in combination with a checkpoint inhibitor. In some embodiments, the checkpoint inhibitor is an anti-PD-1 antibody, an anti-PD-L1 antibody, an anti-CTLA4 antibody, B7-DC-Fc, LAG3, or TIM3. In some embodiments, the checkpoint inhibitor is selected from the group consisting of MEDI0680, AMP-224, nivolumab, pembrolizumab, pidilizumab, MEDI4736, MPDL3280A, ipilimumab and tremelimumab. In some embodiments, the checkpoint inhibitor is pembrolizumab.

In some embodiments, the subject has been previously treated for the prostate cancer or melanoma. In some embodiments, the subject did not respond to the treatment (e.g., the subject experienced disease progression during treatment). In some embodiments, the subject relapsed after the treatment. In some embodiments, the subject experienced disease progression after the treatment. In some embodiments, the treatment previously administered to the subject was not an anti-LIV1 antibody or antigen-binding fragment thereof as described herein.

Certain prostate cancers or melanomas show detectable levels of LIV1 measured at either the protein (e.g., by immunoassay using one of the exemplified antibodies) or the mRNA level. In certain embodiments, a prostate cancer or melanoma shows elevated levels of LIV1 relative to non-cancerous tissue or cells of the same type, e.g., prostate or skin cells or tissues from the same patient. In other embodiments, a prostate cancer or melanoma shows similar levels of LIV1 relative to non-cancerous prostate cancer or melanoma cells of the same type, e.g., from the same patient.

An exemplary level of LIV1 protein on prostate cancer or melanoma cells amenable to treatment is 5,000-150,000 LIV1 proteins per cell, although cancers associated with higher or lower levels can be treated. Optionally, LIV1 levels (e.g., LIV1 protein levels) in a prostate cancer or melanoma from a subject are measured before performing treatment. In some embodiments, at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of the cancer cells express LIV1.

A. Prostate Cancer

Prostate cancer is a form of cancer that develops in the prostate gland. Globally, prostate cancer is the second-most common cancer. It is the fifth-leading cause of cancer-related death in men. In 2018, it was diagnosed in 1.2 million and caused 359,000 deaths. Prostate cancer cells can spread by breaking away from a prostate tumor. They can travel through blood vessels or lymph nodes to reach other parts of the body. After spreading, cancer cells may attach to other tissues and grow to form new tumors, causing damage where they land. Many cases are managed with active surveillance or watchful waiting. Other treatments may include a combination of surgery, radiation therapy, hormone therapy, or chemotherapy. The cancer chemotherapeutic docetaxel has been used as treatment for castration-resistant prostate cancer (CRPC) with a median survival benefit of 2 to 3 months. A second-line chemotherapy treatment is cabazitaxel. A combination of bevacizumab, docetaxel, thalidomide and prednisone appears effective in the treatment of CRPC. Immunotherapy treatment with sipuleucel-T in CRPC increases survival by four months. The second line hormonal therapy abiraterone increases survival by 4.6 months. Enzalutamide is another second line hormonal agent with a five month survival advantage. Both abiraterone and enzalutamide are currently in clinical trials in those with CRPC who have not previously received chemotherapy. More effective treatments for these patients with locally advanced or systemic disease are needed.

The invention provides methods for treating prostate cancer with an anti-LIV1 antibody or antigen-binding fragment thereof or antibody-drug conjugate (e.g., a LIV1-ADC) described herein. In one aspect, the anti-LIV1 antibody or antigen-binding fragment thereof or antibody-drug conjugate (e.g., a LIV1-ADC) described herein are for use in a method of treating prostate cancer in a subject. In some embodiments, the subject has an Eastern Cooperative Oncology Group (ECOG) score of 0 or 1. In some embodiments, the subject has an ECOG score of 0. In some embodiments, the subject has an ECOG score of 1. In some embodiments, the prostate cancer is an adenocarcinoma of the prostate. In some embodiments, the prostate cancer is metastatic. In some embodiments, the prostate cancer is castration resistant prostate cancer. In some embodiments, the subject has not been previously treated with chemotherapy for the castration resistant prostate cancer. In some embodiments, the subject has not been previously treated with radioisotope therapy. In some embodiments, the subject has been previously treated with no more than one line of an androgen-receptor targeted therapy. In some embodiments, the subject has been previously treated with one line of an androgen-receptor targeted therapy. In some embodiments, the subject has not been previously treated with an androgen-receptor targeted therapy. In some embodiments, the androgen-receptor targeted therapy is selected from the group consisting of abiraterone acetate, enzalutamide, apalutamide, and darolutamide. In some embodiments, the androgen-receptor targeted therapy is abiraterone acetate. In some embodiments, the androgen-receptor targeted therapy is enzalutamide. In some embodiments, the androgen-receptor targeted therapy is apalutamide. In some embodiments, the androgen-receptor targeted therapy is darolutamide. In some embodiments, the subject has not been previously treated with chemotherapy to treat the metastatic prostate cancer. In some embodiments, the subject does not have a BRCA mutation. In some embodiments, the subject has been previously treated with one or more therapeutic agents and did not respond to the treatment, wherein the one or more therapeutic agents is not the antibody or antigen-binding fragment thereof. In some embodiments, the subject has been previously treated with one or more therapeutic agents and relapsed after the treatment, wherein the one or more therapeutic agents is not the antibody or antigen-binding fragment thereof. In some embodiments, the subject has been previously treated with one or more therapeutic agents and has experienced disease progression during treatment, wherein the one or more therapeutic agents is not the antibody or antigen-binding fragment thereof. In some embodiments, the cancer is an advanced stage cancer. In some embodiments, the advanced stage cancer is a stage 3 or stage 4 cancer. In some embodiments, the cancer is recurrent cancer. In some embodiments, the cancer is unresectable. In some embodiments, the subject received prior treatment with standard of care therapy for the cancer and failed the prior treatment. In some embodiments, an anti-LIV1 antibody or antigen-binding fragment thereof or antibody-drug conjugate (e.g., a LIV1-ADC) described herein is administered by intravenous infusion. In some embodiments, an anti-LIV1 antibody or antigen-binding fragment thereof or antibody-drug conjugate (e.g., a LIV1-ADC) described herein is administered as a monotherapy. In some embodiments, an anti-LIV1 antibody or antigen-binding fragment thereof or antibody-drug conjugate (e.g., a LIV1-ADC) described herein is administered by intravenous infusion. In some embodiments, an anti-LIV1 antibody or antigen-binding fragment thereof or antibody-drug conjugate (e.g., a LIV1-ADC) described herein is administered in combination with a checkpoint inhibitor. In some embodiments, the checkpoint inhibitor is an anti-PD-1 antibody, an anti-PD-L1 antibody, an anti-CTLA4 antibody, B7-DC-Fc, LAG3, or TIM3. In some embodiments, the checkpoint inhibitor is selected from the group consisting of MEDI0680, AMP-224, nivolumab, pembrolizumab, pidilizumab, MEDI4736, MPDL3280A, ipilimumab and tremelimumab. In some embodiments, the checkpoint inhibitor is pembrolizumab. In a particular embodiment, the subject is a human.

B. Melanoma

Melanoma, also known as malignant melanoma, is a type of skin cancer that develops from the pigment-producing cells known as melanocytes. Melanomas typically occur in the skin but may rarely occur in the mouth, intestines or eye (uveal melanoma). In women, they most commonly occur on the legs, while in men they most commonly occur on the back. About 25% of melanomas develop from moles. Changes in a mole that can indicate melanoma include an increase in size, irregular edges, change in color, itchiness or skin breakdown. Treatment is typically removal by surgery. In those with slightly larger cancers, nearby lymph nodes may be tested for spread (metastasis). Most people are cured if spread has not occurred. For those in whom melanoma has spread, immunotherapy, biologic therapy, radiation therapy or chemotherapy may improve survival. With treatment, the five-year survival rates in the United States are 99% among those with localized disease, 65% when the disease has spread to lymph nodes and 25% among those with distant spread. Melanoma is the most dangerous type of skin cancer. Globally, in 2012, it newly occurred in 232,000 people. In 2015, there were 3.1 million people with active disease, which resulted in 59,800 deaths. Australia and New Zealand have the highest rates of melanoma in the world. There are also high rates in Northern Europe and North America, while it is less common in Asia, Africa and Latin America. In the United States melanoma occurs about 1.6 times more often in men than women. There are four main types of melanoma, superficial spreading melanoma, nodular melanoma, lentigo maligna melanoma, and acral lentiginous melanoma. Chemotherapy drugs such as Dacarbazine have been the backbone of metastatic melanoma treatment since FDA approval in 1975 however, its efficacy in terms of survival has never been proven in a randomized controlled trial. Small-molecule targeted therapies are also used to treat melanoma. The main treatments are BRAF, C-Kit and NRAS inhibitors. BRAF inhibitors, such as vemurafenib and dabrafenib and a MEK inhibitor trametinib are the most effective, approved treatments for BRAF positive melanoma. The therapy combination of dabrafenib and trametinib has a 3-year PFS of 23%, and 5-year PFS of 13%.

The invention provides methods for treating melanoma with an anti-LIV1 antibody or antigen-binding fragment thereof or antibody-drug conjugate (e.g., a LIV1-ADC) described herein. In one aspect, the anti-LIV1 antibody or antigen-binding fragment thereof or antibody-drug conjugate (e.g., a LIV1-ADC) described herein are for use in a method of melanoma in a subject. In some embodiments, the subject has an Eastern Cooperative Oncology Group (ECOG) score of 0 or 1. In some embodiments, the subject has an ECOG score of 0. In some embodiments, the subject has an ECOG score of 1. In some embodiments, the melanoma is metastatic. In some embodiments, the melanoma is locally advanced unresectable melanoma. In some embodiments, the melanoma is locally advanced. In some embodiments, the melanoma is unresectable. In some embodiments, the subject has not been previously treated with cytotoxic chemotherapy. In some embodiments, the subject has been previously treated with no more than 2 prior systemic therapies for advanced disease. In some embodiments, the subject has not previously received systemic therapy for advanced disease. In some embodiments, the subject has been previously treated with 1 prior systemic therapies for advanced disease. In some embodiments, the subject has been previously treated with 2 prior systemic therapies for advanced disease. In some embodiments, the melanoma is cutaneous malignant melanoma. In some embodiments, the melanoma is superficial spreading melanoma. In some embodiments, the melanoma is superficial nodular melanoma. In some embodiments, the melanoma is lentigo maligna melanoma. In some embodiments, the melanoma is acral lentiginous melanoma. In some embodiments, the subject has been previously treated with no more than one line of prior platinum-based cytotoxic therapy. In some embodiments, the subject has not been previously treated with prior platinum-based cytotoxic therapy. In some embodiments, the subject has been previously treated with one line of prior platinum-based cytotoxic therapy. In some embodiments, the subject has been previously treated with an anti-PD-L1 or anti-PD-1 therapy. In some embodiments, the subject has been previously treated with an anti-PD-L1 therapy. In some embodiments, the subject has been previously treated with an anti-PD-1 therapy. In some embodiments, the subject has been previously treated with ipilimumab. In some embodiments, the subject was treated with ipilimumab in combination with the anti-PD-L1 or anti-PD-1 therapy. In some embodiments, the subject has a BRAF mutation. In some embodiments, the subject was treated with a BRAF inhibitor prior to being treated with the anti-PD-L1 or anti-PD-1 therapy. In some embodiments, the subject was treated with a MEK inhibitor. In some embodiments, the subject was treated with a BRAF inhibitor. In some embodiments, the subject was treated with a MEK inhibitor in combination with the BRAF inhibitor. In some embodiments, the subject has been previously treated with one or more therapeutic agents and did not respond to the treatment, wherein the one or more therapeutic agents is not the antibody or antigen-binding fragment thereof. In some embodiments, the subject has been previously treated with one or more therapeutic agents and relapsed after the treatment, wherein the one or more therapeutic agents is not the antibody or antigen-binding fragment thereof. In some embodiments, the subject has been previously treated with one or more therapeutic agents and has experienced disease progression during treatment, wherein the one or more therapeutic agents is not the antibody or antigen-binding fragment thereof. In some embodiments, the cancer is an advanced stage cancer. In some embodiments, the advanced stage cancer is a stage 3 or stage 4 cancer. In some embodiments, the cancer is recurrent cancer. In some embodiments, the cancer is unresectable. In some embodiments, the subject received prior treatment with standard of care therapy for the cancer and failed the prior treatment. In some embodiments, an anti-LIV1 antibody or antigen-binding fragment thereof or antibody-drug conjugate (e.g., a LIV1-ADC) described herein is administered by intravenous infusion. In some embodiments, an anti-LIV1 antibody or antigen-binding fragment thereof or antibody-drug conjugate (e.g., a LIV1-ADC) described herein is administered as a monotherapy. In some embodiments, an anti-LIV1 antibody or antigen-binding fragment thereof or antibody-drug conjugate (e.g., a LIV1-ADC) described herein is administered by intravenous infusion. In some embodiments, an anti-LIV1 antibody or antigen-binding fragment thereof or antibody-drug conjugate (e.g., a LIV1-ADC) described herein is administered in combination with a checkpoint inhibitor. In some embodiments, the checkpoint inhibitor is an anti-PD-1 antibody, an anti-PD-L1 antibody, an anti-CTLA4 antibody, B7-DC-Fc, LAG3, or TIM3. In some embodiments, the checkpoint inhibitor is selected from the group consisting of MEDI0680, AMP-224, nivolumab, pembrolizumab, pidilizumab, MEDI4736, MPDL3280A, ipilimumab and tremelimumab. In some embodiments, the checkpoint inhibitor is pembrolizumab. In a particular embodiment, the subject is a human.

C. Adverse Events

In one aspect, a method of treating cancer, such as solid tumors, e.g., locally advanced or metastatic solid tumors (e.g., prostate cancer and melanoma), with an antibody or antigen-binding fragment thereof or antibody-drug conjugate described herein (e.g., LIV1-ADC), results in the subject developing one or more adverse events. In some embodiments, the subject is administered an additional therapeutic agent to eliminate or reduce the severity of the adverse event. In some embodiments, the one or more adverse events is a grade 1 or greater adverse event. In some embodiments, the one or more adverse events is a grade 2 or greater adverse event. In some embodiments, the one or more adverse events is a grade 3 or greater adverse event. In some embodiments, the one or more adverse events is a grade 1 adverse event. In some embodiments, the one or more adverse events is a grade 2 adverse event. In some embodiments, the one or more adverse events is a grade 3 adverse event. In some embodiments, the one or more adverse events is a grade 4 adverse event. In some embodiments, the one or more adverse events is a serious adverse event. In some of any of the embodiments herein, the subject is administered a treatment with the additional therapeutic agent to eliminate or reduce the severity of the adverse. In some embodiments, the one or more adverse events is a recurrent infusion related reaction and the additional therapeutic agent is an antihistamine, acetaminophen and/or a corticosteroid. In some embodiments, the one or more adverse events is neutropenia and the additional therapeutic agent is growth factor support (e.g., granulocyte-colony stimulating factor [G-CSF]).

In one aspect, a subject treated with an antibody or antigen-binding fragment thereof or antibody-drug conjugate described herein (e.g., LIV1-ADC), is at risk of developing one or more adverse events. In some embodiments, the subject is administered an additional therapeutic agent to prevent the development of the adverse event or to reduce the severity of the adverse event. In some embodiments, the one or more adverse events is a grade 1 or greater adverse event. In some embodiments, the one or more adverse events is a grade 2 or greater adverse event. In some embodiments, the one or more adverse events is a grade 3 or greater adverse event. In some embodiments, the one or more adverse events is a grade 1 adverse event. In some embodiments, the one or more adverse events is a grade 2 adverse event. In some embodiments, the one or more adverse events is a grade 3 adverse event. In some embodiments, the one or more adverse events is a grade 4 adverse event. In some embodiments, the one or more adverse events is a serious adverse event. In some of any of the embodiments herein, the subject is administered a treatment with the additional therapeutic agent to prevent the development of the adverse event or to reduce the severity of the adverse event. In some embodiments, the one or more adverse events is a recurrent infusion related reaction and the additional therapeutic agent is an antihistamine, acetaminophen and/or a corticosteroid. In some embodiments, the one or more adverse events is neutropenia and the additional therapeutic agent is growth factor support (G-CSF).

D. Treatment Outcome

In one aspect, a method of treating a solid tumor, e.g., prostate cancer or melanoma, with an antibody or antigen-binding fragment thereof or antibody-drug conjugate (e.g., LIV1-ADC) as described herein results in an improvement in one or more therapeutic effects in the subject after administration of the antibody or antigen-binding fragment thereof or antibody-drug conjugate (e.g., LIV1-ADC) relative to a baseline. In some embodiments, the one or more therapeutic effects is the size of the tumor derived from the cancer (e.g., prostate cancer or melanoma), the objective response rate, the duration of response, the time to response, progression free survival, overall survival, prostate-specific antigen (PSA) level, PSA duration of response, PSA-PFS, or any combination thereof. In one embodiment, the one or more therapeutic effects is the size of the tumor derived from the cancer (e.g., prostate cancer or melanoma). In one embodiment, the one or more therapeutic effects is decreased tumor size. In one embodiment, the one or more therapeutic effects is stable disease. In one embodiment, the one or more therapeutic effects is partial response. In one embodiment, the one or more therapeutic effects is complete response. In one embodiment, the one or more therapeutic effects is the objective response rate. In one embodiment, the one or more therapeutic effects is the duration of response. In one embodiment, the one or more therapeutic effects is the time to response. In one embodiment, the one or more therapeutic effects is progression free survival. In one embodiment, the one or more therapeutic effects is overall survival. In one embodiment, the one or more therapeutic effects is PSA level. In one embodiment, the one or more therapeutic effects is cancer regression.

In one embodiment of the methods or uses or product for uses provided herein, response to treatment with an antibody or antigen-binding fragment thereof or antibody-drug conjugate as described herein, such as e.g., a LIV1-ADC, may include the following criteria (RECIST Criteria 1.1):

| | Category | Criteria |
|---|---|---|
| Based on | Complete | Disappearance of all target lesions. Any pathological |
| target lesions | Response (CR) | lymph nodes must have reduction in short axis to <10 |
| | | mm. |
| | Partial Response | ≥30% decrease in the sum of the longest diameter |
| | (PR) | (LD) of target lesions, taking as reference the baseline |
| | | sum of LDs. |
| | Stable Disease | Neither sufficient shrinkage to qualify for PR nor |
| | (SD) | sufficient increase to qualify for PD, taking as |
| | | reference the smallest sum of LDs while in trial. |
| | Progressive | ≥20% (and ≥5 mm) increase in the sum of the LDs of |
| | Disease (PD) | target lesions, taking as reference the smallest sum of |
| | | the target LDs recorded while in trial or the appearance |
| | | of one or more new lesions. |
| Based on non- | CR | Disappearance of all non-target lesions and |
| target lesions | | normalization of tumor marker level. All lymph nodes |
| | | must be non-pathological in size (<10 mm short axis). |
| | SD | Persistence of one or more non-target lesion(s) or/and |
| | | maintenance of tumor marker level above the normal |
| | | limits. |
| | PD | Appearance of one or more new lesions and/or |
| | | unequivocal progression of existing non-target lesions. |

In one embodiment of the methods or uses or product for uses provided herein, the effectiveness of treatment with an antibody or antigen-binding fragment thereof or antibody-drug conjugate as described herein, such as e.g., a LIV1-ADC, is assessed by measuring the objective response rate. In some embodiments, the objective response rate is the proportion of patients with tumor size reduction of a predefined amount and for a minimum period of time. In some embodiments the objective response rate is based upon RECIST v1.1. In one embodiment, the objective response rate is at least about 20%, at least about 25%, at least about 30%, at least about 35%, at least about 40%, at least about 45%, at least about 50%, at least about 60%, at least about 70%, or at least about 80%. In one embodiment, the objective response rate is at least about 20%-80%. In one embodiment, the objective response rate is at least about 30%-80%. In one embodiment, the objective response rate is at least about 40%-80%. In one embodiment, the objective response rate is at least about 50%-80%. In one embodiment, the objective response rate is at least about 60%-80%. In one embodiment, the objective response rate is at least about 70%-80%. In one embodiment, the objective response rate is at least about 80%. In one embodiment, the objective response rate is at least about 85%. In one embodiment, the objective response rate is at least about 90%. In one embodiment, the objective response rate is at least about 95%. In one embodiment, the objective response rate is at least about 98%. In one embodiment, the objective response rate is at least about 99%. In one embodiment, the objective response rate is at least 20%, at least 25%, at least 30%, at least 35%, at least 40%, at least 45%, at least 50%, at least 60%, at least 70%, or at least 80%. In one embodiment, the objective response rate is at least 20%-80%. In one embodiment, the objective response rate is at least 30%-80%. In one embodiment, the objective response rate is at least 40%-80%. In one embodiment, the objective response rate is at least 50%-80%. In one embodiment, the objective response rate is at least 60%-80%. In one embodiment, the objective response rate is at least 70%-80%. In one embodiment, the objective response rate is at least 80%. In one embodiment, the objective response rate is at least 85%. In one embodiment, the objective response rate is at least 90%. In one embodiment, the objective response rate is at least 95%. In one embodiment, the objective response rate is at least 98%. In one embodiment, the objective response rate is at least 99%. In one embodiment, the objective response rate is 100%.

In one embodiment of the methods or uses or product for uses provided herein, response to treatment with an antibody or antigen-binding fragment thereof or antibody-drug conjugate as described herein, such as e.g., a LIV1-ADC, is assessed by measuring the size of a tumor derived from the cancer (e.g., prostate cancer or melanoma). In one embodiment, the size of a tumor derived from the cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of the antibody or antigen-binding fragment thereof or antibody-drug conjugate described herein (e.g., a LIV1-ADC). In one embodiment, the size of a tumor derived from the cancer is reduced by at least about 10%-80%. In one embodiment, the size of a tumor derived from the cancer is reduced by at least about 20%-80%. In one embodiment, the size of a tumor derived from the cancer is reduced by at least about 30%-80%. In one embodiment, the size of a tumor derived from the cancer is reduced by at least about 40%-80%. In one embodiment, the size of a tumor derived from the cancer is reduced by at least about 50%-80%. In one embodiment, the size of a tumor derived from the cancer is reduced by at least about 60%-80%. In one embodiment, the size of a tumor derived from the cancer is reduced by at least about 70%-80%. In one embodiment, the size of a tumor derived from the cancer is reduced by at least about 80%. In one embodiment, the size of a tumor derived from the cancer is reduced by at least about 85%. In one embodiment, the size of a tumor derived from the cancer is reduced by at least about 90%. In one embodiment, the size of a tumor derived from the cancer is reduced by at least about 95%. In one embodiment, the size of a tumor derived from the cancer is reduced by at least about 98%. In one embodiment, the size of a tumor derived from the cancer is reduced by at least about 99%. In one embodiment, the size of a tumor derived from the cancer is reduced by at least 10%, at least 15%, at least 20%, at least 25%, at least 30%, at least 35%, at least 40%, at least 45%, at least 50%, at least 60%, at least 70%, or at least 80% relative to the size of the tumor derived from the cancer before administration of the antibody or antigen-binding fragment thereof or antibody-drug conjugate described herein (e.g., a LIV1-ADC). In one embodiment, the size of a tumor derived from the cancer is reduced by at least 10%-80%. In one embodiment, the size of a tumor derived from the cancer is reduced by at least 20%-80%. In one embodiment, the size of a tumor derived from the cancer is reduced by at least 30%-80%. In one embodiment, the size of a tumor derived from the cancer is reduced by at least 40%-80%. In one embodiment, the size of a tumor derived from the cancer is reduced by at least 50%-80%. In one embodiment, the size of a tumor derived from the cancer is reduced by at least 60%-80%. In one embodiment, the size of a tumor derived from the cancer is reduced by at least 70%-80%. In one embodiment, the size of a tumor derived from the cancer is reduced by at least 80%. In one embodiment, the size of a tumor derived from the cancer is reduced by at least 85%. In one embodiment, the size of a tumor derived from the cancer is reduced by at least 90%. In one embodiment, the size of a tumor derived from the cancer is reduced by at least 95%. In one embodiment, the size of a tumor derived from the cancer is reduced by at least 98%. In one embodiment, the size of a tumor derived from the cancer is reduced by at least 99%. In one embodiment, the size of a tumor derived from the cancer is reduced by 100%. In one embodiment, the size of a tumor derived from the cancer is measured by magnetic resonance imaging (MRI). In one embodiment, the size of a tumor derived from the cancer is measured by computed tomography (CT). In one embodiment, the size of a tumor derived from the cancer is measured by positron emission tomography (PET). In one embodiment, the size of a tumor derived from the cancer is measured by ultrasound.

In one embodiment of the methods or uses or product for uses provided described herein, response to treatment with an antibody or antigen-binding fragment thereof or antibody-drug conjugate described herein (e.g., a LIV1-ADC), promotes regression of a tumor derived from the cancer (e.g., prostate cancer or melanoma). In one embodiment, a tumor derived from the cancer regresses by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of the antibody or antigen-binding fragment thereof or antibody-drug conjugate described herein (e.g., a LIV1-ADC). In one embodiment, a tumor derived from the cancer regresses by at least about 10% to about 80%. In one embodiment, a tumor derived from the cancer regresses by at least about 20% to about 80%. In one embodiment, a tumor derived from the cancer regresses by at least about 30% to about 80%. In one embodiment, a tumor derived from the cancer regresses by at least about 40% to about 80%. In one embodiment, a tumor derived from the cancer regresses by at least about 50% to about 80%. In one embodiment, a tumor derived from the cancer regresses by at least about 60% to about 80%. In one embodiment, a tumor derived from the cancer regresses by at least about 70% to about 80%. In one embodiment, a tumor derived from the cancer regresses by at least about 80%. In one embodiment, a tumor derived from the cancer regresses by at least about 85%. In one embodiment, a tumor derived from the cancer regresses by at least about 90%. In one embodiment, a tumor derived from the cancer regresses by at least about 95%. In one embodiment, a tumor derived from the cancer regresses by at least about 98%. In one embodiment, a tumor derived from the cancer regresses by at least about 99%. In one embodiment, a tumor derived from the cancer regresses by at least 10%, at least 15%, at least 20%, at least 25%, at least 30%, at least 35%, at least 40%, at least 45%, at least 50%, at least 60%, at least 70%, or at least 80% relative to the size of the tumor derived from the cancer before administration of the antibody or antigen-binding fragment thereof or antibody-drug conjugate described herein (e.g., a LIV1-ADC). In one embodiment, a tumor derived from the cancer regresses by at least 10% to 80%. In one embodiment, a tumor derived from the cancer regresses by at least 20% to 80%. In one embodiment, a tumor derived from the cancer regresses by at least 30% to 80%. In one embodiment, a tumor derived from the cancer regresses by at least 40% to 80%. In one embodiment, a tumor derived from the cancer regresses by at least 50% to 80%. In one embodiment, a tumor derived from the cancer regresses by at least 60% to 80%. In one embodiment, a tumor derived from the cancer regresses by at least 70% to 80%. In one embodiment, a tumor derived from the cancer regresses by at least 80%. In one embodiment, a tumor derived from the cancer regresses by at least 85%. In one embodiment, a tumor derived from the cancer regresses by at least 90%. In one embodiment, a tumor derived from the cancer regresses by at least 95%. In one embodiment, a tumor derived from the cancer regresses by at least 98%. In one embodiment, a tumor derived from the cancer regresses by at least 99%. In one embodiment, a tumor derived from the cancer regresses by 100%. In one embodiment, regression of a tumor is determined by measuring the size of the tumor by magnetic resonance imaging (MRI). In one embodiment, regression of a tumor is determined by measuring the size of the tumor by computed tomography (CT). In one embodiment, regression of a tumor is determined by measuring the size of the tumor by positron emission tomography (PET). In one embodiment, regression of a tumor is determined by measuring the size of the tumor by ultrasound.

In one embodiment of the methods or uses or product for uses described herein, response to treatment with an antibody or antigen-binding fragment thereof or antibody-drug conjugate described herein (e.g., a LIV1-ADC), is assessed by measuring the time of progression free survival after administration of the antibody or antigen-binding fragment thereof or antibody-drug conjugate described herein (e.g., a LIV1-ADC). In some embodiments, the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody or antigen-binding fragment thereof or antibody-drug conjugate described herein (e.g., a LIV1-ADC). In some embodiments, the subject exhibits progression-free survival of at least about 6 months after administration of the antibody or antigen-binding fragment thereof or antibody-drug conjugate described herein (e.g., a LIV1-ADC). In some embodiments, the subject exhibits progression-free survival of at least about one year after administration of the antibody or antigen-binding fragment thereof or antibody-drug conjugate described herein (e.g., a LIV1-ADC). In some embodiments, the subject exhibits progression-free survival of at least about two years after administration of the antibody or antigen-binding fragment thereof or antibody-drug conjugate described herein (e.g., a LIV1-ADC). In some embodiments, the subject exhibits progression-free survival of at least about three years after administration of the antibody or antigen-binding fragment thereof or antibody-drug conjugate described herein (e.g., a LIV1-ADC). In some embodiments, the subject exhibits progression-free survival of at least about four years after administration of the antibody or antigen-binding fragment thereof or antibody-drug conjugate described herein (e.g., a LIV1-ADC). In some embodiments, the subject exhibits progression-free survival of at least about five years after administration of the antibody or antigen-binding fragment thereof or antibody-drug conjugate described herein (e.g., a LIV1-ADC). In some embodiments, the subject exhibits progression-free survival of at least 1 month, at least 2 months, at least 3 months, at least 4 months, at least 5 months, at least 6 months, at least 7 months, at least 8 months, at least 9 months, at least 10 months, at least 11 months, at least 12 months, at least eighteen months, at least two years, at least three years, at least four years, or at least five years after administration of the antibody or antigen-binding fragment thereof or antibody-drug conjugate described herein (e.g., a LIV1-ADC). In some embodiments, the subject exhibits progression-free survival of at least 6 months after administration of the antibody or antigen-binding fragment thereof or antibody-drug conjugate described herein (e.g., a LIV1-ADC). In some embodiments, the subject exhibits progression-free survival of at least one year after administration of the antibody or antigen-binding fragment thereof or antibody-drug conjugate described herein (e.g., a LIV1-ADC). In some embodiments, the subject exhibits progression-free survival of at least two years after administration of the antibody or antigen-binding fragment thereof or antibody-drug conjugate described herein (e.g., a LIV1-ADC). In some embodiments, the subject exhibits progression-free survival of at least three years after administration of the antibody or antigen-binding fragment thereof or antibody-drug conjugate described herein (e.g., a LIV1-ADC). In some embodiments, the subject exhibits progression-free survival of at least four years after administration of the antibody or antigen-binding fragment thereof or antibody-drug conjugate described herein (e.g., a LIV1-ADC). In some embodiments, the subject exhibits progression-free survival of at least five years after administration of the antibody or antigen-binding fragment thereof or antibody-drug conjugate described herein (e.g., a LIV1-ADC).

In one embodiment of the methods or uses or product for uses described herein, response to treatment with an antibody or antigen-binding fragment thereof or antibody-drug conjugate described herein (e.g., a LIV1-ADC), is assessed by measuring the time of overall survival after administration of the antibody or antigen-binding fragment thereof or antibody-drug conjugate described herein (e.g., a LIV1-ADC). In some embodiments, the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody or antigen-binding fragment thereof or antibody-drug conjugate described herein (e.g., a LIV1-ADC). In some embodiments, the subject exhibits overall survival of at least about 6 months after administration of the antibody or antigen-binding fragment thereof or antibody-drug conjugate described herein (e.g., a LIV1-ADC). In some embodiments, the subject exhibits overall survival of at least about one year after administration of the antibody or antigen-binding fragment thereof or antibody-drug conjugate described herein (e.g., a LIV1-ADC). In some embodiments, the subject exhibits overall survival of at least about two years after administration of the antibody or antigen-binding fragment thereof or antibody-drug conjugate described herein (e.g., a LIV1-ADC). In some embodiments, the subject exhibits overall survival of at least about three years after administration of the antibody or antigen-binding fragment thereof or antibody-drug conjugate described herein (e.g., a LIV1-ADC). In some embodiments, the subject exhibits overall survival of at least about four years after administration of the antibody or antigen-binding fragment thereof or antibody-drug conjugate described herein (e.g., a LIV1-ADC). In some embodiments, the subject exhibits overall survival of at least about five years after administration of the antibody or antigen-binding fragment thereof or antibody-drug conjugate described herein (e.g., a LIV1-ADC). In some embodiments, the subject exhibits overall survival of at least 1 month, at least 2 months, at least 3 months, at least 4 months, at least 5 months, at least 6 months, at least 7 months, at least 8 months, at least 9 months, at least 10 months, at least 11 months, at least 12 months, at least eighteen months, at least two years, at least three years, at least four years, or at least five years after administration of the antibody or antigen-binding fragment thereof or antibody-drug conjugate described herein (e.g., a LIV1-ADC). In some embodiments, the subject exhibits overall survival of at least 6 months after administration of the antibody or antigen-binding fragment thereof or antibody-drug conjugate described herein (e.g., a LIV1-ADC). In some embodiments, the subject exhibits overall survival of at least one year after administration of the antibody or antigen-binding fragment thereof or antibody-drug conjugate described herein (e.g., a LIV1-ADC). In some embodiments, the subject exhibits overall survival of at least two years after administration of the antibody or antigen-binding fragment or antibody-drug conjugate thereof described herein (e.g., a LIV1-ADC). In some embodiments, the subject exhibits overall survival of at least three years after administration of the antibody or antigen-binding fragment thereof or antibody-drug conjugate described herein (e.g., a LIV1-ADC). In some embodiments, the subject exhibits overall survival of at least four years after administration of the antibody or antigen-binding fragment thereof or antibody-drug conjugate described herein (e.g., a LIV1-ADC). In some embodiments, the subject exhibits overall survival of at least five years after administration of the antibody or antigen-binding fragment thereof described herein (e.g., a LIV1-ADC).

In one embodiment of the methods or uses or product for uses described herein, response to treatment with an antibody or antigen-binding fragment thereof or antibody-drug conjugate described herein (e.g., a LIV1-ADC), is assessed by measuring the duration of response to the antibody or antigen-binding fragment thereof or antibody-drug conjugate described herein (e.g., a LIV1-ADC) after administration of the antibody or antigen-binding fragment thereof or antibody-drug conjugate described herein (e.g., a LIV1-ADC). In some embodiments, the duration of response to the antibody or antigen-binding fragment thereof or antibody-drug conjugate described herein (e.g., a LIV1-ADC) is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody or antigen-binding fragment thereof or antibody-drug conjugate described herein (e.g., a LIV1-ADC). In some embodiments, the duration of response to the antibody or antigen-binding fragment thereof or antibody-drug conjugate described herein (e.g., a LIV1-ADC) is at least about 6 months after administration of the antibody or antigen-binding fragment thereof or antibody-drug conjugate described herein (e.g., a LIV1-ADC). In some embodiments, the duration of response to anti-LIV1 antibody or antigen-binding fragment thereof or antibody-drug conjugate described herein (e.g., a LIV1-ADC) is at least about one year after administration of the anti-LIV1 antibody or antigen-binding fragment thereof or antibody-drug conjugate described herein (e.g., a LIV1-ADC). In some embodiments, the duration of response to the antibody or antigen-binding fragment thereof or antibody-drug conjugate described herein (e.g., a LIV1-ADC) is at least about two years after administration of the antibody or antigen-binding fragment thereof or antibody-drug conjugate described herein (e.g., a LIV1-ADC). In some embodiments, the duration of response to the anti-LIV1 antibody or antigen-binding fragment thereof or antibody-drug conjugate described herein (e.g., a LIV1-ADC) is at least about three years after administration of the antibody or antigen-binding fragment thereof or antibody-drug conjugate described herein (e.g., a LIV1-ADC). In some embodiments, the duration of response to the antibody or antigen-binding fragment thereof or antibody-drug conjugate described herein (e.g., a LIV1-ADC) is at least about four years after administration of the antibody or antigen-binding fragment thereof or antibody-drug conjugate described herein (e.g., a LIV1-ADC). In some embodiments, the duration of response to the antibody or antigen-binding fragment thereof or antibody-drug conjugate described herein (e.g., a LIV1-ADC) is at least about five years after administration of the antibody or antigen-binding fragment thereof or antibody-drug conjugate described herein (e.g., a LIV1-ADC). In some embodiments, the duration of response to the antibody or antigen-binding fragment thereof or antibody-drug conjugate described herein (e.g., a LIV1-ADC) is at least 1 month, at least 2 months, at least 3 months, at least 4 months, at least 5 months, at least 6 months, at least 7 months, at least 8 months, at least 9 months, at least 10 months, at least 11 months, at least 12 months, at least eighteen months, at least two years, at least three years, at least four years, or at least five years after administration of the antibody or antigen-binding fragment thereof or antibody-drug conjugate described herein (e.g., a LIV1-ADC). In some embodiments, the duration of response to the antibody or antigen-binding fragment thereof or antibody-drug conjugate described herein (e.g., a LIV1-ADC) is at least 6 months after administration of the antibody or antigen-binding fragment thereof or antibody-drug conjugate described herein (e.g., a LIV1-ADC). In some embodiments, the duration of response to the antibody or antigen-binding fragment thereof or antibody-drug conjugate described herein (e.g., a LIV1-ADC) is at least one year after administration of the antibody or antigen-binding fragment thereof or antibody-drug conjugate described herein (e.g., a LIV1-ADC). In some embodiments, the duration of response to the antibody or antigen-binding fragment thereof or antibody-drug conjugate described herein (e.g., a LIV1-ADC) is at least two years after administration of the antibody or antigen-binding fragment thereof or antibody-drug conjugate described herein (e.g., a LIV1-ADC). In some embodiments, the duration of response to the antibody or antigen-binding fragment thereof or antibody-drug conjugate described herein (e.g., a LIV1-ADC) is at least three years after administration of the antibody or antigen-binding fragment thereof or antibody-drug conjugate described herein (e.g., a LIV1-ADC). In some embodiments, the duration of response to the antibody or antigen-binding fragment thereof or antibody-drug conjugate described herein (e.g., a LIV1-ADC) is at least four years after administration of the antibody or antigen-binding fragment thereof or antibody-drug conjugate described herein (e.g., a LIV1-ADC). In some embodiments, the duration of response to the antibody or antigen-binding fragment thereof or antibody-drug conjugate described herein (e.g., a LIV1-ADC) is at least five years after administration of the antibody or antigen-binding fragment thereof or antibody-drug conjugate described herein (e.g., a LIV1-ADC).

In one embodiment of the methods or uses or product for uses described herein, response to treatment with an antibody or antigen-binding fragment thereof or antibody-drug conjugate described herein (e.g., a LIV1-ADC), is assessed by measuring the PSA level in a subject with prostate cancer. In some embodiments, the PSA level is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the PSA level before administration of the antibody or antigen-binding fragment thereof or antibody-drug conjugate described herein (e.g., a LIV1-ADC). In some embodiments, the PSA level is reduced by at least about 10% relative to the PSA level before administration of the antibody or antigen-binding fragment thereof or antibody-drug conjugate described herein (e.g., a LIV1-ADC). In some embodiments, the PSA level is reduced by at least about 15% relative to the PSA level before administration of the antibody or antigen-binding fragment thereof or antibody-drug conjugate described herein (e.g., a LIV1-ADC). In some embodiments, the PSA level is reduced by at least about 20% relative to the PSA level before administration of the antibody or antigen-binding fragment thereof or antibody-drug conjugate described herein (e.g., a LIV1-ADC). In some embodiments, the PSA level is reduced by at least about 25% relative to the PSA level before administration of the antibody or antigen-binding fragment thereof or antibody-drug conjugate described herein (e.g., a LIV1-ADC). In some embodiments, the PSA level is reduced by at least about 30% relative to the PSA level before administration of the antibody or antigen-binding fragment thereof or antibody-drug conjugate described herein (e.g., a LIV1-ADC). In some embodiments, the PSA level is reduced by at least about 35% relative to the PSA level before administration of the antibody or antigen-binding fragment thereof or antibody-drug conjugate described herein (e.g., a LIV1-ADC). In some embodiments, the PSA level is reduced by at least about 40% relative to the PSA level before administration of the antibody or antigen-binding fragment thereof or antibody-drug conjugate described herein (e.g., a LIV1-ADC). In some embodiments, the PSA level is reduced by at least about 45% relative to the PSA level before administration of the antibody or antigen-binding fragment thereof or antibody-drug conjugate described herein (e.g., a LIV1-ADC). In some embodiments, the PSA level is reduced by at least about 50% relative to the PSA level before administration of the antibody or antigen-binding fragment thereof or antibody-drug conjugate described herein (e.g., a LIV1-ADC). In some embodiments, the PSA level is reduced by at least about 55% relative to the PSA level before administration of the antibody or antigen-binding fragment thereof or antibody-drug conjugate described herein (e.g., a LIV1-ADC). In some embodiments, the PSA level is reduced by at least about 60% relative to the PSA level before administration of the antibody or antigen-binding fragment thereof or antibody-drug conjugate described herein (e.g., a LIV1-ADC). In some embodiments, the PSA level is reduced by at least about 65% relative to the PSA level before administration of the antibody or antigen-binding fragment thereof or antibody-drug conjugate described herein (e.g., a LIV1-ADC). In some embodiments, the PSA level is reduced by at least about 70% relative to the PSA level before administration of the antibody or antigen-binding fragment thereof or antibody-drug conjugate described herein (e.g., a LIV1-ADC). In some embodiments, the PSA level is reduced by at least about 75% relative to the PSA level before administration of the antibody or antigen-binding fragment thereof or antibody-drug conjugate described herein (e.g., a LIV1-ADC). In some embodiments, the PSA level is reduced by at least about 80% relative to the PSA level before administration of the antibody or antigen-binding fragment thereof or antibody-drug conjugate described herein (e.g., a LIV1-ADC).

In one embodiment of the methods or uses or product for uses described herein, response to treatment with an antibody or antigen-binding fragment thereof or antibody-drug conjugate described herein (e.g., a LIV1-ADC), is assessed by measuring the time of PSA progression free survival after administration of the antibody or antigen-binding fragment thereof or antibody-drug conjugate described herein (e.g., a LIV1-ADC). In some embodiments, PSA progression free survival is defined as the time from the start of study treatment to first occurrence of PSA progression or death, whichever comes first. In some embodiments, PSA progression is defined in prostate cancer subjects with a PSA decline from baseline an increase of ≥25% and at least 2 ng/mL over nadir value, confirmed by second PSA value at least 3 weeks later. In some embodiments, PSA progression is defined in prostate cancer subjects with no PSA decline from baseline as an increase in PSA by 25% (at least 2 ng/mL) above baseline level, confirmed by second PSA value at least 3 weeks apart. In some embodiments, the subject exhibits PSA progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody or antigen-binding fragment thereof or antibody-drug conjugate described herein (e.g., a LIV1-ADC). In some embodiments, the subject exhibits PSA progression-free survival of at least about 6 months after administration of the antibody or antigen-binding fragment thereof or antibody-drug conjugate described herein (e.g., a LIV1-ADC). In some embodiments, the subject exhibits PSA progression-free survival of at least about one year after administration of the antibody or antigen-binding fragment thereof or antibody-drug conjugate described herein (e.g., a LIV1-ADC). In some embodiments, the subject exhibits PSA progression-free survival of at least about two years after administration of the antibody or antigen-binding fragment thereof or antibody-drug conjugate described herein (e.g., a LIV1-ADC). In some embodiments, the subject exhibits PSA progression-free survival of at least about three years after administration of the antibody or antigen-binding fragment thereof or antibody-drug conjugate described herein (e.g., a LIV1-ADC). In some embodiments, the subject exhibits PSA progression-free survival of at least about four years after administration of the antibody or antigen-binding fragment thereof or antibody-drug conjugate described herein (e.g., a LIV1-ADC). In some embodiments, the subject exhibits PSA progression-free survival of at least about five years after administration of the antibody or antigen-binding fragment thereof or antibody-drug conjugate described herein (e.g., a LIV1-ADC). In some embodiments, the subject exhibits PSA progression-free survival of at least 1 month, at least 2 months, at least 3 months, at least 4 months, at least 5 months, at least 6 months, at least 7 months, at least 8 months, at least 9 months, at least 10 months, at least 11 months, at least 12 months, at least eighteen months, at least two years, at least three years, at least four years, or at least five years after administration of the antibody or antigen-binding fragment thereof or antibody-drug conjugate described herein (e.g., a LIV1-ADC). In some embodiments, the subject exhibits PSA progression-free survival of at least 6 months after administration of the antibody or antigen-binding fragment thereof or antibody-drug conjugate described herein (e.g., a LIV1-ADC). In some embodiments, the subject exhibits PSA progression-free survival of at least one year after administration of the antibody or antigen-binding fragment thereof or antibody-drug conjugate described herein (e.g., a LIV1-ADC). In some embodiments, the subject exhibits PSA progression-free survival of at least two years after administration of the antibody or antigen-binding fragment thereof or antibody-drug conjugate described herein (e.g., a LIV1-ADC). In some embodiments, the subject exhibits PSA progression-free survival of at least three years after administration of the antibody or antigen-binding fragment thereof or antibody-drug conjugate described herein (e.g., a LIV1-ADC). In some embodiments, the subject exhibits PSA progression-free survival of at least four years after administration of the antibody or antigen-binding fragment thereof or antibody-drug conjugate described herein (e.g., a LIV1-ADC). In some embodiments, the subject exhibits PSA progression-free survival of at least five years after administration of the antibody or antigen-binding fragment thereof or antibody-drug conjugate described herein (e.g., a LIV1-ADC).

In one embodiment of the methods or uses or product for uses described herein, response to treatment with an antibody or antigen-binding fragment thereof or antibody-drug conjugate described herein (e.g., a LIV1-ADC), is assessed by measuring the PSA duration of response to the antibody or antigen-binding fragment thereof or antibody-drug conjugate described herein (e.g., a LIV1-ADC) after administration of the antibody or antigen-binding fragment thereof or antibody-drug conjugate described herein (e.g., a LIV1-ADC). In some embodiments, the PSA duration of response to the antibody or antigen-binding fragment thereof or antibody-drug conjugate described herein (e.g., a LIV1-ADC) is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody or antigen-binding fragment thereof or antibody-drug conjugate described herein (e.g., a LIV1-ADC). In some embodiments, the PSA duration of response to the antibody or antigen-binding fragment thereof or antibody-drug conjugate described herein (e.g., a LIV1-ADC) is at least about 6 months after administration of the antibody or antigen-binding fragment thereof or antibody-drug conjugate described herein (e.g., a LIV1-ADC). In some embodiments, the PSA duration of response to anti-LIV1 antibody or antigen-binding fragment thereof or antibody-drug conjugate described herein (e.g., a LIV1-ADC) is at least about one year after administration of the anti-LIV1 antibody or antigen-binding fragment thereof or antibody-drug conjugate described herein (e.g., a LIV1-ADC). In some embodiments, the PSA duration of response to the antibody or antigen-binding fragment thereof or antibody-drug conjugate described herein (e.g., a LIV1-ADC) is at least about two years after administration of the antibody or antigen-binding fragment thereof or antibody-drug conjugate described herein (e.g., a LIV1-ADC). In some embodiments, the PSA duration of response to the anti-LIV1 antibody or antigen-binding fragment thereof or antibody-drug conjugate described herein (e.g., a LIV1-ADC) is at least about three years after administration of the antibody or antigen-binding fragment thereof or antibody-drug conjugate described herein (e.g., a LIV1-ADC). In some embodiments, the PSA duration of response to the antibody or antigen-binding fragment thereof or antibody-drug conjugate described herein (e.g., a LIV1-ADC) is at least about four years after administration of the antibody or antigen-binding fragment thereof or antibody-drug conjugate described herein (e.g., a LIV1-ADC). In some embodiments, PSA the duration of response to the antibody or antigen-binding fragment thereof or antibody-drug conjugate described herein (e.g., a LIV1-ADC) is at least about five years after administration of the antibody or antigen-binding fragment thereof or antibody-drug conjugate described herein (e.g., a LIV1-ADC). In some embodiments, the PSA duration of response to the antibody or antigen-binding fragment thereof or antibody-drug conjugate described herein (e.g., a LIV1-ADC) is at least 1 month, at least 2 months, at least 3 months, at least 4 months, at least 5 months, at least 6 months, at least 7 months, at least 8 months, at least 9 months, at least 10 months, at least 11 months, at least 12 months, at least eighteen months, at least two years, at least three years, at least four years, or at least five years after administration of the antibody or antigen-binding fragment thereof or antibody-drug conjugate described herein (e.g., a LIV1-ADC). In some embodiments, the PSA duration of response to the antibody or antigen-binding fragment thereof or antibody-drug conjugate described herein (e.g., a LIV1-ADC) is at least 6 months after administration of the antibody or antigen-binding fragment thereof or antibody-drug conjugate described herein (e.g., a LIV1-ADC). In some embodiments, the PSA duration of response to the antibody or antigen-binding fragment thereof or antibody-drug conjugate described herein (e.g., a LIV1-ADC) is at least one year after administration of the antibody or antigen-binding fragment thereof or antibody-drug conjugate described herein (e.g., a LIV1-ADC). In some embodiments, the PSA duration of response to the antibody or antigen-binding fragment thereof or antibody-drug conjugate described herein (e.g., a LIV1-ADC) is at least two years after administration of the antibody or antigen-binding fragment thereof or antibody-drug conjugate described herein (e.g., a LIV1-ADC). In some embodiments, the PSA duration of response to the antibody or antigen-binding fragment thereof or antibody-drug conjugate described herein (e.g., a LIV1-ADC) is at least three years after administration of the antibody or antigen-binding fragment thereof or antibody-drug conjugate described herein (e.g., a LIV1-ADC). In some embodiments, the PSA duration of response to the antibody or antigen-binding fragment thereof or antibody-drug conjugate described herein (e.g., a LIV1-ADC) is at least four years after administration of the antibody or antigen-binding fragment thereof or antibody-drug conjugate described herein (e.g., a LIV1-ADC). In some embodiments, the PSA duration of response to the antibody or antigen-binding fragment thereof or antibody-drug conjugate described herein (e.g., a LIV1-ADC) is at least five years after administration of the antibody or antigen-binding fragment thereof or antibody-drug conjugate described herein (e.g., a LIV1-ADC).

### IV. PHARMACEUTICAL COMPOSITIONS AND FORMULATIONS

For therapeutic use, an antibody or antigen-binding fragment thereof or antibody-drug conjugate (e.g., a LIV1-ADC) is combined with a pharmaceutically acceptable carrier. As used herein, “pharmaceutically acceptable carrier” means buffers, carriers, and excipients suitable for use in contact with the tissues of human beings and animals without excessive toxicity, irritation, allergic response, or other problem or complication, commensurate with a reasonable benefit/risk ratio. The carrier(s) should be “acceptable” in the sense of being compatible with the other ingredients of the formulations and not deleterious to the recipient. Pharmaceutically acceptable carriers include buffers, solvents, dispersion media, coatings, isotonic and absorption delaying agents, and the like, that are compatible with pharmaceutical administration. The use of such media and agents for pharmaceutically active substances is known in the art.

Accordingly, antibody or antigen-binding fragment thereof or antibody-drug conjugate (e.g., a LIV1-ADC) compositions of the present invention can comprise at least one of any suitable excipients, such as, but not limited to, diluent, binder, stabilizer, buffers, salts, lipophilic solvents, preservative, adjuvant or the like. Pharmaceutically acceptable excipients are preferred. Non-limiting examples of, and methods of preparing such sterile solutions are well known in the art, such as, but not limited to, those described in Gennaro, Ed., Remington's Pharmaceutical Sciences, 18th Edition, Mack Publishing Co. (Easton, Pa.) 1990. Pharmaceutically acceptable carriers can be routinely selected that are suitable for the mode of administration, solubility and/or stability of the antibody molecule, fragment or variant composition as well known in the art or as described herein.

Suitable pharmaceutical excipients and/or additives for use in the antibody molecule compositions according to the invention are known in the art, e.g., as listed in “Remington: The Science & Practice of Pharmacy,” 19th ed., Williams & Williams, (1995), and in the “Physician's Desk Reference,” 52nd ed., Medical Economics, Montvale, N.J. (1998).

Pharmaceutical compositions containing an antibody or antigen-binding fragment thereof or antibody-drug conjugate (e.g., a LIV1-ADC) as disclosed herein can be presented in a dosage unit form and can be prepared by any suitable method. A pharmaceutical composition should be formulated to be compatible with its intended route of administration. Examples of routes of administration are intravenous (IV), intradermal, inhalation, transdermal, topical, transmucosal, and rectal administration. A preferred route of administration for monoclonal antibodies is IV infusion. Useful formulations can be prepared by methods known in the pharmaceutical art. For example, see Remington's Pharmaceutical Sciences (1990) supra. Formulation components suitable for parenteral administration include a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor EL™ (BASF, Parsippany, N.J.) or phosphate buffered saline (PBS). The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyethylene glycol), and suitable mixtures thereof.

Pharmaceutical formulations are preferably sterile. Sterilization can be accomplished by any suitable method, e.g., filtration through sterile filtration membranes. Where the composition is lyophilized, filter sterilization can be conducted prior to or following lyophilization and reconstitution.

The compositions of this invention may be in a variety of forms. These include, for example, liquid, semi-solid and solid dosage forms, such as liquid solutions (e.g., injectable and infusible solutions), dispersions or suspensions, and liposomes. The particular form depends on the intended mode of administration and therapeutic application. In exemplary embodiments, compositions provided are in the form of injectable or infusible solutions. Exemplary administration is parenteral (e.g., intravenous, subcutaneous, intraocular, intraperitoneal, intramuscular). In an exemplary embodiment, the preparation is administered by intravenous infusion or injection. In another preferred embodiment, the preparation is administered by intramuscular or subcutaneous injection.

The phrases “parenteral administration” and “administered parenterally” as used herein means modes of administration other than enteral and topical administration, usually by injection, and includes, without limitation, intravenous, intramuscular, subcutaneous, intraarterial, intrathecal, intracapsular, intraorbital, intravitreous, intracardiac, intradermal, intraperitoneal, transtracheal, inhaled, subcutaneous, subcuticular, intraarticular, subcapsular, subarachnoid, intraspinal, epidural and intrasternal injection and infusion.

Exemplary dosages of an antibody or antigen-binding fragment thereof or antibody-drug conjugate (e.g., a LIV1-ADC) are about 0.5 mg/kg of a subject's body weight, about 0.75 mg/kg of a subject's body weight, about 1.0 mg/kg of a subject's body weight, about 1.25 mg/kg of a subject's body weight, about 1.5 mg/kg of a subject's body weight, about 1.67 mg/kg of a subject's body weight, about 1.75 mg/kg of a subject's body weight, about 2.0 mg/kg of a subject's body weight, about 2.25 mg/kg of a subject's body weight, about 2.5 mg/kg of a subject's body weight, about 2.75 mg/kg of a subject's body weight, or about 2.8 mg/kg of a subject's body weight. In a particular embodiment, an exemplary dose of LIV1-ADC is about 0.75 mg/kg of a subject's body weight. In another particular embodiment, an exemplary dose of LIV1-ADC is about 1.0 mg/kg of a subject's body weight. In another particular embodiment, an exemplary dose of LIV1-ADC is about 1.25 mg/kg of a subject's body weight. In another particular embodiment, an exemplary dose of LIV1-ADC is about 1.5 mg/kg of a subject's body weight. In another particular embodiment, an exemplary dose of LIV1-ADC is about 1.67 mg/kg of a subject's body weight. In another particular embodiment, an exemplary dose of LIV1-ADC is about 1.75 mg/kg of a subject's body weight. In another particular embodiment, an exemplary dose of LIV1-ADC is about 2.0 mg/kg of a subject's body weight. In another particular embodiment, an exemplary dose of LIV1-ADC is about 2.25 mg/kg of a subject's body weight. In another particular embodiment, an exemplary dose of LIV1-ADC is about 2.5 mg/kg of a subject's body weight. In another particular embodiment, an exemplary dose of LIV1-ADC is about 2.75 mg/kg of a subject's body weight. In another particular embodiment, an exemplary dose of LIV1-ADC is about 2.8 mg/kg of a subject's body weight. In another particular embodiment, a maximum exemplary dose of LIV1-ADC is about 100 mg per cycle. In another particular embodiment, a maximum exemplary dose of LIV1-ADC is about 125 mg per cycle. In another particular embodiment, a maximum exemplary dose of LIV1-ADC is about 200 mg per cycle. In another particular embodiment, a maximum exemplary dose of LIV1-ADC is about 250 mg per cycle.

In certain exemplary embodiments, a subject is administered a dose of about 2.5 mg/kg, at a maximum dose of about 200 mg, once every three weeks. In certain exemplary embodiments, a subject is administered an intravenous dose of about 2.5 mg/kg, at a maximum dose of about 200 mg, once every three weeks.

In certain exemplary embodiments, a subject is administered a dose of about 2.5 mg/kg, at a maximum dose of about 250 mg, once every three weeks. In certain exemplary embodiments, a subject is administered an intravenous dose of about 2.5 mg/kg, at a maximum dose of about 250 mg, once every three weeks.

In certain exemplary embodiments, a subject is administered a dose of about 1.0 mg/kg, at a maximum dose of about 100 mg, once every one week. In certain exemplary embodiments, a subject is administered an intravenous dose of about 1.0 mg/kg, at a maximum dose of about 100 mg, once every one week.

In certain exemplary embodiments, a subject is administered a dose of about 1.25 mg/kg, at a maximum dose of about 125 mg, once every one week. In certain exemplary embodiments, a subject is administered an intravenous dose of about 1.25 mg/kg, at a maximum dose of about 125 mg, once every one week.

In certain exemplary embodiments, a subject is administered a dose of about 1.0 mg/kg, at a maximum dose of about 200 mg, once every one week. In certain exemplary embodiments, a subject is administered an intravenous dose of about 1.0 mg/kg, at a maximum dose of about 200 mg, once every one week.

In certain exemplary embodiments, a subject is administered a dose of about 1.25 mg/kg, at a maximum dose of about 200 mg, once every one week. In certain exemplary embodiments, a subject is administered an intravenous dose of about 1.25 mg/kg, at a maximum dose of about 200 mg, once every one week.

In certain exemplary embodiments, a subject is administered a dose of about 2.5 mg/kg, at a maximum dose of about 250 mg, once every three weeks. In certain exemplary embodiments, a subject is administered an intravenous dose of about 2.5 mg/kg, at a maximum dose of about 250 mg, once every three weeks. In certain exemplary embodiments, the subject is further administered G-CSF. In certain exemplary embodiments, if the anti-LIV1 antibody or antigen-binding fragment thereof (e.g., a LIV1-ADC) is used at a dose of greater than or equal to about 200 mg and less than or equal to about 250 mg once every three weeks, the subject is further administered G-CSF. In certain exemplary embodiments, if the anti-LIV1 antibody or antigen-binding fragment thereof (e.g., a LIV1-ADC) is used at a dose of greater than or equal to 200 mg and less than or equal to 250 mg once every three weeks, the subject is further administered G-CSF. In certain embodiments, the G-CSF is administered prophylactically. In certain embodiments, the G-CSF is recombinant human G-CSF. In certain embodiments, the G-CSF is filgrastim (NEUPOGEN®). In certain embodiments, the G-CSF is PEG-filgrastim (NEULASTA®). In certain embodiments, the G-CSF is lenograstim (GRANOCYTE®). In certain embodiments, the G-CSF is tbo-filgrastim (GRANIX®).

In certain exemplary embodiments, a subject is administered a dose of about 1.0 mg/kg, at a maximum dose of about 100 mg, once every one week. In certain exemplary embodiments, a subject is administered an intravenous dose of about 1.0 mg/kg, at a maximum dose of about 100 mg, once every one week. In certain exemplary embodiments, the subject is further administered G-CSF. In certain exemplary embodiments, if the anti-LIV1 antibody or antigen-binding fragment thereof (e.g., a LIV1-ADC) is used at a dose of greater than or equal to about 80 mg and less than or equal to about 100 mg once every one week, the subject is further administered G-CSF. In certain exemplary embodiments, if the anti-LIV1 antibody or antigen-binding fragment thereof (e.g., a LIV1-ADC) is used at a dose of greater than or equal to 80 mg and less than or equal to 100 mg once every one week, the subject is further administered G-CSF. In certain embodiments, the G-CSF is administered prophylactically. In certain embodiments, the G-CSF is recombinant human G-CSF. In certain embodiments, the G-CSF is filgrastim (NEUPOGEN®). In certain embodiments, the G-CSF is PEG-filgrastim (NEULASTA®). In certain embodiments, the G-CSF is lenograstim (GRANOCYTE®). In certain embodiments, the G-CSF is tbo-filgrastim (GRANIX®).

In certain exemplary embodiments, a subject is administered a dose of about 1.25 mg/kg, at a maximum dose of about 125 mg, once every one week. In certain exemplary embodiments, a subject is administered an intravenous dose of about 1.25 mg/kg, at a maximum dose of about 125 mg, once every one week. In certain exemplary embodiments, the subject is further administered G-CSF. In certain exemplary embodiments, if the anti-LIV1 antibody or antigen-binding fragment thereof (e.g., a LIV1-ADC) is used at a dose of greater than or equal to about 100 mg and less than or equal to about 125 mg once every one week, the subject is further administered G-CSF. In certain exemplary embodiments, if the anti-LIV1 antibody or antigen-binding fragment thereof (e.g., a LIV1-ADC) is used at a dose of greater than or equal to 100 mg and less than or equal to 125 mg once every one week, the subject is further administered G-CSF. In certain embodiments, the G-CSF is administered prophylactically. In certain embodiments, the G-CSF is recombinant human G-CSF. In certain embodiments, the G-CSF is filgrastim (NEUPOGEN®). In certain embodiments, the G-CSF is PEG-filgrastim (NEULASTA®). In certain embodiments, the G-CSF is lenograstim (GRANOCYTE®). In certain embodiments, the G-CSF is tbo-filgrastim (GRANIX®).

In certain exemplary embodiments, a subject is administered LIV1-ADC at a dose of about 0.75 mg/kg of body weight once every 7 days (±1 day). In certain exemplary embodiments, a subject is administered LIV1-ADC at a dose of about 1.0 mg/kg of body weight once every 7 days (±1 day). In certain exemplary embodiments, a subject is administered LIV1-ADC at a dose of about 1.25 mg/kg of body weight once every 7 days (±1 day). In certain exemplary embodiments, a subject is administered LIV1-ADC at a dose of about 1.5 mg/kg of body weight once every 7 days (±1 day). In certain exemplary embodiments, a subject is administered LIV1-ADC at a dose of about 1.67 mg/kg of body weight once every 7 days (±1 day).

In certain exemplary embodiments, the subject is further administered G-CSF. In certain exemplary embodiments, if the anti-LIV1 antibody or antigen-binding fragment thereof or antibody-drug conjugate (e.g., a LIV1-ADC) is used at a dose of greater than or equal to about 200 mg and less than or equal to about 250 mg, the subject is further administered G-CSF. In certain exemplary embodiments, if the anti-LIV1 antibody or antigen-binding fragment thereof or antibody-drug conjugate (e.g., a LIV1-ADC) is used at a dose of greater than or equal to 200 mg and less than or equal to 250 mg, the subject is further administered G-CSF. In certain embodiments, the G-CSF is administered prophylactically. In certain embodiments, the G-CSF is recombinant human G-CSF. In certain embodiments, the G-CSF is filgrastim (NEUPOGEN®). In certain embodiments, the G-CSF is PEG-filgrastim (NEULASTA®). In certain embodiments, the G-CSF is lenograstim (GRANOCYTE®). In certain embodiments, the G-CSF is tbo-filgrastim (GRANIX®).

The present invention provides a kit, comprising packaging material and at least one vial comprising a solution of at least one an antibody or antigen-binding fragment thereof or antibody-drug conjugate (e.g., a LIV1-ADC) with the prescribed buffers and/or preservatives, optionally in an aqueous diluent. The concentration of preservative used in the formulation is a concentration sufficient to yield an anti-microbial effect. Such concentrations are dependent on the preservative selected and are readily determined by the skilled artisan.

Various delivery systems can be used to administer antibodies or antigen-binding fragments thereof or antibody-drug conjugate to a subject. In certain exemplary embodiments, administration of an antibody or antigen-binding fragment thereof or antibody-drug conjugate (e.g., a LIV1-ADC) is by intravenous infusion.

Any of the formulations described above can be stored in a liquid or frozen form and can be optionally subjected to a preservation process. In some embodiments, the formulations described above are lyophilized, i.e., they are subjected to lyophilization. In some embodiments, the formulations described above are subjected to a preservation process, for example, lyophilization, and are subsequently reconstituted with a suitable liquid, for example, water. By lyophilized, it is meant that the composition has been freeze-dried under a vacuum. Lyophilization typically is accomplished by freezing a particular formulation such that the solutes are separated from the solvent(s). The solvent is then removed by sublimation (i.e., primary drying) and next by desorption (i.e., secondary drying).

The formulations of the present invention can be used with the methods described herein or with other methods for treating disease. The antibody or antigen-binding fragment thereof or antibody-drug conjugate (e.g., LIV1-ADC) formulations may be further diluted before administration to a subject. In some embodiments, the formulations will be diluted with saline and held in IV bags or syringes before administration to a subject. Accordingly, in some embodiments, the methods for treating a cancer, such as a LIV1-expressing cancer, in a subject will comprise administering to a subject in need thereof a weekly dose of a pharmaceutical composition comprising an antibody or antigen-binding fragment thereof or antibody-drug conjugate (e.g., a LIV1-ADC).

### V. ARTICLES OF MANUFACTURE AND KITS

In another aspect, an article of manufacture or kit is provided which comprises an antibody or antigen-binding fragment thereof or antibody-drug conjugate described herein (e.g., a LIV1-ADC). The article of manufacture or kit may further comprise instructions for use of the antibody or antigen-binding fragment thereof or antibody-drug conjugate described herein (e.g., a LIV1-ADC) in the methods of the invention. Thus, in certain embodiments, the article of manufacture or kit comprises instructions for the use of an anti-LIV1 antibody or antigen-binding fragment thereof described herein (e.g., a LIV1-ADC) in methods for treating cancer (e.g., prostate cancer or melanoma) in a subject comprising administering to the subject an effective amount of an anti-LIV1 antibody or antigen-binding fragment thereof or antibody-drug conjugate described herein (e.g., a LIV1-ADC). In some embodiments the cancer is a locally advanced cancer. In some embodiments, the cancer is a metastatic cancer. In some embodiments, the cancer is a prostate cancer as described herein. In some embodiments, the cancer is a melanoma as described herein. In some embodiments, the subject is a human.

The article of manufacture or kit may further comprise a container. Suitable containers include, for example, bottles, vials (e.g., dual chamber vials), syringes (such as single or dual chamber syringes) and test tubes. In some embodiments, the container is a vial. The container may be formed from a variety of materials such as glass or plastic. The container holds the formulation.

The article of manufacture or kit may further comprise a label or a package insert, which is on or associated with the container, may indicate directions for reconstitution and/or use of the formulation. The label or package insert may further indicate that the formulation is useful or intended for subcutaneous, intravenous (e.g., intravenous infusion), or other modes of administration for treating cancer, e.g., prostate cancer or melanoma, as described herein in a subject. The container holding the formulation may be a single-use vial or a multi-use vial, which allows for repeat administrations of the reconstituted formulation. The article of manufacture or kit may further comprise a second container comprising a suitable diluent. The article of manufacture or kit may further include other materials desirable from a commercial, therapeutic, and user standpoint, including other buffers, diluents, filters, needles, syringes, and package inserts with instructions for use.

The article of manufacture or kit herein optionally further comprises a container comprising a second medicament, wherein an anti-LIV1 antibody or antigen-binding fragment thereof or antibody-drug conjugate described herein (e.g., a LIV1-ADC) is a first medicament, and which article or kit further comprises instructions on the label or package insert for treating the subject with the second medicament, in an effective amount. In some embodiments, the label or package insert indicates that the first and second medicaments are to be administered sequentially or simultaneously, as described herein. In some embodiments, the label or package insert indicates that the first medicament is to be administered prior to the administration of the second medicament. In some embodiments, the label or package insert indicates that second medicament is to be administered prior to the first medicament.

The article of manufacture or kit herein optionally further comprises a container comprising a second medicament, wherein the second medicament is for eliminating or reducing the severity of one or more adverse events, wherein an antibody or antigen-binding fragment thereof or antibody-drug conjugate described herein (e.g., a LIV1-ADC) is a first medicament, and which article or kit further comprises instructions on the label or package insert for treating the subject with the second medicament, in an effective amount. In some embodiments, the label or package insert indicates that the first and second medicaments are to be administered sequentially or simultaneously, as described herein. In some embodiments, the label or package insert indicates that the first medicament is to be administered prior to the administration of the second medicament. In some embodiments, the label or package insert indicates that second medicament is to be administered prior to the first medicament.

In some embodiments, an antibody or antigen-binding fragment thereof or antibody-drug conjugate described herein (e.g., a LIV1-ADC) is present in the container as a lyophilized powder. In some embodiments, the lyophilized powder is in a hermetically sealed container, such as a vial, an ampoule or sachette, indicating the quantity of the active agent. Where the pharmaceutical is administered by injection, an ampoule of sterile water for injection or saline can be, for example, provided, optionally as part of the kit, so that the ingredients can be mixed prior to administration. Such kits can further include, if desired, one or more of various conventional pharmaceutical components, such as, for example, containers with one or more pharmaceutically acceptable carriers, additional containers, etc., as will be readily apparent to those skilled in the art. Printed instructions, either as inserts or as labels, indicating quantities of the components to be administered, guidelines for administration, and/or guidelines for mixing the components can also be included in the kit.

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing and method steps.

It will be readily apparent to those skilled in the art that other suitable modifications and adaptations of the methods described herein may be made using suitable equivalents without departing from the scope of the embodiments disclosed herein. Having now described certain embodiments in detail, the same will be more clearly understood by reference to the following examples, which are included for purposes of illustration only and are not intended to be limiting. All patents, patent applications and references described herein are incorporated by reference in their entireties for all purposes.

### EXAMPLES

### Example 1: A Phase II Study of Ladiratuzumab Vedotin in Advanced Prostate Cancer and Melanoma

Ladiratuzumab vedotin (LV) is an antibody-drug conjugate comprising a LIV targeted human monoclonal immunoglobulin conjugated via a protease-cleavable valine citrulline linker to the drug monomethyl auristatin E (MMAE), a dolastatin 10 analog. Dolastatins and auristatins belong to a class of chemotherapies that act as microtubule disrupting agents.

This study evaluates the efficacy, safety and tolerability of 1.0 mg/kg or 1.25 mg/kg ladiratuzumab vedotin in patients with locally advanced or metastatic prostate cancer or melanoma. Patients with locally-advanced or metastatic prostate cancer or melanoma whose disease has progressed after first and subsequent lines of treatment have significant unmet medical need for therapies that can meaningfully improve their prognosis. Prostate cancer and melanoma are promising candidates for treatment with ladiratuzumab vedotin based on the characteristics shown in the following table.

TABLE 3
Melanoma and prostate cancer characteristics

| Key considerations | Melanoma | Prostate |
|---|---|---|
| LIV1 expression | 67-92% | 99-100% |
| Role of LIV1 in cancer | No data | + |
| progression and metastasis | | |
| MMAE ADC sensitive | − | − |
| | (ETBR, GV) | (PSMA, STEAP1, |
| | | SLC44A) |
| Predicted MMAE sensitivity | + | + |
| Vinca sensitive | − | − |
| Eribulin/maytansine sensitive | No data | No data |
| Prior microtubule inhibitor | No | 1L hormone |
| exposure | | refractory |
| Population size | 17k | 49k |
| Front-line development | ++ | − |

Multiple MMAE ADCs have been evaluated in prostate cancer and melanoma, mostly on a Q3W schedule. Anti-PSMA, anti-SLC44A4, anti-STEAP1, and anti-tissue factor ADCs have been tested in metastatic prostate cancer and yielded objective response rates ranging from 0% to 10%. Anti-ETBR and anti-gpNNB ADCs have been tested in melanoma and yielded objective response rates of 11%.

### Methods

This global, open label, multicenter trial is designed to assess the safety, tolerability, and activity of ladiratuzumab vedotin for the treatment of selected prostate cancer and melanoma. Eligible patients are at least 18 years of age with unresectable, locally advanced or metastatic cancer. Patients are enrolled into one of 2 cohorts based on tumor type, including prostate cancer and melanoma. There will be no biomarker pre-selection of patients based on LIV1 expression.

In all eligible patients, ladiratuzumab vedotin is administered at a dose of 1.0 mg/kg or 1.25 mg/kg as a 30 minute intravenous infusion on Day 1 of each 7-day cycle (Q1W). Dosing is based on the subject's body weight, measured each cycle. The maximum dose will be 200 mg per infusion. Response is assessed every 7 weeks (±7 days) for the first 24 weeks and every 12 weeks (±7 days) thereafter. For prostate cancer, Prostate Cancer Clinical Trials Working Group 3 (PCWG3) modified RECIST v1.1 is used by the investigator to score responses for primary and secondary endpoints as well as progression. For melanoma, RECIST v1.1 is used by the investigator to score responses for primary and secondary endpoints as well as progression. Objective responses are confirmed with repeat scans 4-6 weeks after the first documentation of response. For prostate cancer subjects, a bone scan should be performed to assess disease at baseline. For subjects with prostate cancer, prostate-specific antigen (PSA) will also be assessed every 3 weeks (±3 days), at the end of treatment, and every 12 weeks (±7 days) thereafter. PSA response is defined as a reduction from the baseline PSA level of at least 50% confirmed ≥3 weeks apart. Imaging assessments (bone scan/CT/MRI) will be performed every 8-9 weeks (±3 days) for the first 24 weeks, then every 12 weeks (±7 days) thereafter. Safety will be assessed throughout the study, and adverse event (AE) severity will be graded using the National Cancer Institute Common Terminology Criteria for Adverse Events (NCI CTCAE), version 5.

The primary analysis of the study will be performed separately for each cohort when all treated subjects in the cohort have been followed for at least 6 months or come off study, whichever comes first. The primary efficacy endpoint of confirmed ORR per RECIST v1.1 will be estimated for each cohort based on the full analysis set (FAS), comprising all subjects who received any amount of study treatment. The point estimate of ORR and 90% exact confidence intervals (CIs) using the Clopper-Pearson method will be provided for each cohort.

Interim futility analyses will be performed separately for each cohort after at least 12 subjects of a given cohort have been treated and are efficacy evaluable post-baseline. The Bayesian predictive probability approach will be used to determine the futility criteria. At the time of each interim analysis, the predictive probability of success (PPoS) will be calculated. A PPoS <10% indicates that it is unlikely the ORR will be better than the response rate of current standard of care at the end of the study given the interim result. Based on efficacy and safety data, together with the PPoS, a cohort may be stopped early by the sponsor.

Inclusion criteria and exclusion criteria for patients enrolled in trial are shown in Table 4.

TABLE 4
List of inclusion and exclusion criteria

| | |
|---|---|
| Inclusion | 1. Age of at least 18 years, or legal age according to local regulations, |
| Criteria | whichever is older |
| | 2. Measurable disease according to RECIST v1.1 as assessed by the |
| | investigator |
| | A minimum of one non-nodal lesion ≥10 mm in the longest diameter |
| | from a non-irradiated area; or |
| | Lymph node lesion ≥15 mm in the shortest diameter from a non- |
| | irradiated area |
| | If target lesion(s) are located within previously irradiated area only, the |
| | subject can be enrolled only if there has been demonstrated progression |
| | in the “in field” lesion and upon approval of the sponsor's medical |
| | monitor |
| | 3. Eastern Cooperative Oncology Group (ECOG) Performance Score of 0 or 1 |
| | 4. The following baseline laboratory data: |
| | absolute neutrophil count (ANC) ≥1500/μL assessed at least 2 weeks |
| | after growth factor support, if applicable |
| | platelet count ≥100 × 10⁹/L assessed at least 2 weeks after transfusion |
| | with blood products, if applicable |
| | hemoglobin (≥8.0 g/dL) assessed at least 2 weeks after transfusion with |
| | blood products and/or growth factor support, if applicable |
| | serum bilirubin ≤1.5 × upper limit of normal (ULN) or direct bilirubin ≤3 × ULN |
| | in subjects diagnosed with Gilbert's syndrome |
| | estimated glomerular filtration rate (GFR) ≥30 mL/min/1.73 m2 using |
| | the Modification of Diet in Renal Disease (MDRD) study equation as |
| | applicable |
| | alanine aminotransferase (ALT) and aspartate aminotransferase (AST) ≤1.5 × ULN |
| | (if liver metastases are present, then ≤3 × ULN is allowed) |
| | 5. Subjects of childbearing potential, under the following conditions: |
| | a. Must have a negative serum or urine pregnancy test (minimum |
| | sensitivity 25 mIU/mL or equivalent units of beta human chorionic |
| | gonadotropin [β-hCG]) result within 3 days prior to the first dose of LV. |
| | Subjects with false positive results and documented verification that the |
| | subject is not pregnant are eligible for participation. |
| | b. Must agree not to try to become pregnant during the study and for at |
| | least 6 months after the final dose of study drug administration |
| | c. Must agree not to breastfeed or donate ova, starting at time of informed |
| | consent and continuing through 6 months after the final dose of study |
| | drug administration |
| | d. If sexually active in a way that could lead to pregnancy, must |
| | consistently use 2 highly effective methods of birth control starting at |
| | time of informed consent and continuing throughout the study and for at |
| | least 6 months after the final dose of study drug administration |
| | 6. Subjects who can father children, under the following conditions: |
| | a. Must agree not to donate sperm starting at time of informed consent and |
| | continuing throughout the study period and for at least 6 months after |
| | the final study drug administration |
| | b. If sexually active with a subject of childbearing potential in a way that |
| | could lead to pregnancy, must consistently use 2 highly effective |
| | methods of birth control starting at time of informed consent and |
| | continuing throughout the study and for at least 6 months after the final |
| | dose of study drug administration |
| | c. If sexually active with a subject who is pregnant or breastfeeding, must |
| | consistently use one of 2 contraception options starting at time of |
| | informed consent and continuing throughout the study and for at least |
| | 6 months after the final dose of study drug administration |
| | 7. Available and adequate archival baseline tumor sample is required. If an |
| | archival baseline tumor sample is not available, a fresh biopsy sample may |
| | be submitted if medically feasible or the medical monitor should be |
| | contacted to review this requirement. Formalin fixed paraffin embedded |
| | (FFPE) blocks and core needle or excisional biopsy of metastatic site are |
| | preferred. |
| | 8. Relapsed, locally advance or metastatic prostate cancer or melanoma that has |
| | failed prior lines of systemic treatment as specified and which are not |
| | candidates for standard therapy. |
| | Castration-resistant prostate cancer |
| | i. Must have histologically or cytologically confirmed |
| | adenocarcinoma of the prostate: |
| | a. Subjects with components of small cell or neuroendocrine |
| | histology are excluded. |
| | ii. Must have metastatic castration-resistant disease: |
| | a. Castration-resistant is defined as progressive disease |
| | following adequate medical or surgical (bilateral |
| | orchiectomy) castration and testosterone <50 ng/dL (<2.0 |
| | nM). |
| | iii. Must have been ≥28 days between cessation of androgen receptor- |
| | targeted therapy and start of study treatment; |
| | iv. Must have received no more than 1 prior line of androgen receptor- |
| | targeted therapy (eg, abiraterone acetate, enzalutamide, |
| | apalutamide, or darolutamide) for metastatic castration-sensitive |
| | prostate cancer (CSPC) or CRPC; |
| | v. No prior cytotoxic chemotherapy in the metastatic CRPC setting: |
| | a. For subjects who received cytotoxic chemotherapy for |
| | CSPC, at least 6 months must have elapsed between last |
| | dose of chemotherapy and start of study treatment; |
| | b. No more than 1 prior line of cytotoxic chemotherapy for |
| | CSPC |
| | vi. Subjects with measurable and non-measurable disease according to |
| | PCWG3 are eligible if the following criteria are met: |
| | a. A minimum starting PSA level ≥1.0 ng/mL; |
| | b. Subjects with measurable soft tissue disease must have |
| | evidence of measurable soft tissue disease according to |
| | PCWG3 criteria. Previously normal (<1.0 cm) lymph nodes |
| | must have grown by ≥5 mm in the short axis to be |
| | considered to have progressed; |
| | c. Subjects with non-measurable disease (enrollment after |
| | interim analysis and no more than 15 subjects in total) must |
| | have documented rising PSA levels or appearance of new |
| | lesion according to PCWG3: |
| | PSA progression as defined by 2 increases in PSA |
| | level over a previous reference value (a minimum of |
| | 3 PSA values total) with an interval of ≥1 week |
| | between each assessment where the PSA value at |
| | screening should be ≥1 ng/mL, OR |
| | Bone disease progression defined by 2 or more new |
| | lesions on bone scan |
| | vii. Subjects with known breast cancer gene (BRCA) mutations are |
| | excluded; |
| | viii. No prior radioisotope therapy or radiotherapy to ≥30% of bone |
| | marrow. |
| | Melanoma |
| | i. Must have histologically or cytologically confirmed cutaneous |
| | malignant melanoma; |
| | a. Subjects with mucosal, acral, or uveal melanoma are |
| | excluded. |
| | ii. Must have locally advanced unresectable or metastatic stage |
| | disease; |
| | iii. Must have measurable disease: |
| | a. Lesions that have had prior intralesional therapies including |
| | talimogene laherparepvec (T-VEC) are not considered |
| | measurable; |
| | b. Subjects with metastases limited to the skin (skin-only |
| | metastasis) are excluded. |
| | iv. Must have progressive disease following anti-PD(L)l therapy: |
| | a. Prior ipilimumab therapy is allowed; |
| | b. May have received BRAF +/− MEK inhibitor therapy if |
| | BRAF mutated; |
| | c. No prior cytotoxic chemotherapy in the advanced disease |
| | setting; |
| | d. No more than 2 prior systemic therapies for advanced |
| | disease; |
| | e. Adjuvant therapy within 6 months of diagnosis of advanced |
| | disease is considered as 1 line of therapy |
| Exclusion | 1. Active concurrent malignancy or a previous malignancy within the past 3 |
| Criteria | years |
| | Exception: malignancies with a negligible risk of metastasis or death |
| | (e.g., 5-year OS ≥ 90%) |
| | 2. Known active central nervous system lesions (including leptomeningeal |
| | metastasis) that have not been definitively treated |
| | 3. Any ongoing clinically significant toxicity associated with prior treatment |
| | (Grade 2 or higher) |
| | 4. Ongoing sensory or motor neuropathy of Grade ≥2 |
| | 5. Has received prior radiotherapy within 2 weeks of start of study treatment |

LV is a sterile, preservative-free, white to off-white lyophilized cake or powder for reconstitution for IV administration. LV is supplied in single-use glass vials. Each drug product vial contains LV for injection, trehalose, histidine, and polysorbate 80. Drug product vials are labeled with a nominal content of 40 mg/vial. Each vial contains 45 mg of LV. Enough overfill is included to allow for 40 mg of LV to be withdrawn for use.

When reconstituted with 8.8 mL water for injection (WFI), United States Pharmacopeia (USP) grade or equivalent, the concentration of reconstituted LV product is 5 mg/mL. The reconstituted drug product is a clear to slightly opalescent, colorless to light yellow solution with no visible particulate matter. The pH is approximately 6.0. The reconstituted solution is subsequently diluted in sterile 0.9% Sodium Chloride for Injection, USP grade or equivalent, for IV administration.

Dose modifications for LV treatment-associated toxicity are described in Table 5. The maximum doses after modifications are:
100 mg for subjects reduced to 1.0 mg/kg
75 mg for subjects reduced to 0.75 mg/kg

TABLE 5
Dose levels

| Dose Level | Dose | |
|---|---|---|
| Starting dose | 1.25 | mg/kg |
| −1 | 1.0 | mg/kg |
| −2 | 0.75 | mg/kg |

Doses reduced for LV-related toxicity should not be re-escalated.

If a subject has a clinically significant, unresolved AE on Day 1 of Cycle 2 (C2) or beyond, the start of the cycle may be delayed.

In the event a subject is unable to tolerate their dose level, additional treatment cycles (C2 or later) may be administered at a lower dose level upon approval by the medical monitor.

Objectives and endpoints are described in Table 6. Confirmed Objective Response Rate (ORR) is defined as the proportion of subjects who achieve a confirmed Complete Response (CR) or Partial Response (PR) according to RECIST v1.1 as assessed by the investigator. For prostate cancer, confirmed Objective Response Rate (ORR) is defined as the proportion of subjects who achieve a confirmed Complete Response (CR) or Partial Response (PR) according to PCWG3 modified RECIST v1.1 as assessed by the investigator. Subjects who do not have at least 2 post-baseline response assessments (initial response and confirmation scan) will be counted as non-responders. For metastatic, castration-resistant prostate cancer (mCRPC) patients, soft tissue tumor assessment by computed tomography or magnetic resonance imaging scan (CT/MRI) and bone scans according to PCWG3 (modified RECIST v1.1 criteria specific to prostate cancer).

Confirmed PSA response rate is defined as the proportion of subjects with a reduction from baseline PSA level of at least 50%, measured twice ≥3 weeks apart. PSA response rate will be assessed per PCWG3.

Disease Control Rate (DCR) is defined as the proportion of subjects who achieve a confirmed CR or PR according to RECIST v1.1 as assessed by the investigator, or meet the SD criteria at least once after start of study treatment at a minimum interval of 6 weeks. Subjects who do not have at least 1 post-baseline response assessment will be counted as non-responders.

Duration of Response (DOR) is defined as the time from the first documentation of objective response (CR or PR that is subsequently confirmed) to the first documentation of PD or death due to any cause, whichever comes first.

DOR data will be censored as described below:
Subjects who do not have PD and are still on study at the time of an analysis will be censored at the date of last disease assessment documenting absence of PD.
Subjects who started a new anticancer treatment prior to documentation of PD will be censored at the date of last disease assessment prior to the start of new treatment.
Subjects who are removed from the study prior to documentation of PD will be censored at the date of last disease assessment documenting absence of PD.
DOR will only be calculated for subjects who achieve a confirmed CR or PR.

Progression-free survival (PFS) is defined as the time from the start of study treatment to the first documentation of PD by RECIST v1.2 or clinical PD or by PSA progression (prostate cancer cohort) or death due to any cause, whichever comes first.

The same censoring rules as for DOR will be applied to PFS. Subjects lacking an evaluation of tumor response after their first dose of study drug will have their event time censored at Day 1.

Overall Survival (OS) is defined as the time from the start of study treatment to date of death due to any cause. In the absence of death, survival time will be censored at the last date the subject is known to be alive (i.e., date of last contact).

Prostate serum antigen progression-free survival (PSA-PFS) is defined as the time from the start of study treatment to first occurrence of PSA progression or death, whichever comes first. PSA progression is defined as:
1. In subjects with PSA decline from baseline: increase of ≥25% and at least 2 ng/mL over nadir value, confirmed by second PSA value at least 3 weeks later;
2. In subjects with no PSA decline from baseline:
   a. increase in PSA by 25% (at least 2 ng/mL) above baseline level, confirmed by second PSA value at least 3 weeks apart. Early rise in PSA only indicated progression if it was associated with another sign of disease progression or if it continued beyond 12 weeks.

TABLE 6
Objectives and endpoints

| Primary Objective | Corresponding Primary Endpoint |
|---|---|
| Evaluate antitumor activity of LV | Investigator-determined confirmed ORR as |
| | measured by RECIST v1.1 for all tumor types |
| | For prostate cancer, investigator-determined PSA |
| | response in addition to ORR |
| Secondary Objectives | Corresponding Secondary Endpoints |
| Evaluate the safety and tolerability of LV | Type, incidence, severity, seriousness, and |
| | relatedness of AEs |
| Evaluate stability and control of disease | Investigator-determined DCR as measured by |
| | RECIST v1.1 |
| Evaluate durability of response in subjects who | Investigator-determined DOR as measured by |
| respond to LV | RECIST v1.1 for all tumors |
| | For prostate cancer, investigator-determined time |
| | to PSA DOR |
| Evaluate PFS of subjects treated with LV | Investigator-determined PFS as measured by |
| | RECIST v1.1 for all tumors |
| | For prostate cancer, investigator-determined PS A- |
| | PFS |
| Evaluate survival of subjects treated with LV | OS |
| Assess PK of LV | Selected PK parameters for LV, total antibody, |
| | and MMAE |
| Assess immunogenicity of LV | Incidence of antitherapeutic antibodies to LV |
| Assess antitherapeutic antibody response to LV | Incidence of antitherapeutic antibodies to LV |
| Additional Objectives | Corresponding Additional Endpoints |
| Assess biomarkers of biological activity and | Relationship between biomarkers in blood and |
| resistance and predictive biomarkers of response | tumor tissue to efficacy, safety, or other |
| | biomarker endpoints following treatment with LV |

For the prostate cancer cohort, the confirmed PSA response rate and 90% exact confidence interval (CI) will also be presented.

### Example 2: Anti-Tumor Activity of Ladiratuzumab Vedotin in Patients with Prostate Cancer

Human patients with unresectable, locally advanced or metastatic prostate cancer will be treated with ladiratuzumab vedotin (LV). Patients will have castration-resistant prostate cancer and have previously received androgen-receptor-targeted therapy. Patients will not have received prior chemotherapy for metastatic castration resistant prostate

LV will be administered by intravenous (IV) infusion at a dose of 1.0 mg/kg or 1.25 mg/kg on Day 1 of each 7-day cycle. Dosing may not exceed 200 mg per infusion. An individual's dose may be modified based upon treatment-related AEs. Tumor assessment according to Prostate Cancer Clinical Trials Working Group 3 (PCWG3) modified RECIST v1.1 will be performed every 7 weeks (±7 days) for the first 24 weeks and then every 12 weeks (±7 days) thereafter. Objective responses will be confirmed with repeat scans 4-6 weeks after the first documentation of response. LV will be administered at a dose of 1.0 mg/kg or 1.25 mg/kg as a 30 minute IV infusion every 7 days.

For prostate cancer subjects, PSA will be assessed every 3 weeks (±3 days). Soft tissue tumor assessment by computed tomography or magnetic resonance imaging scan (CT/MRI) and bone scans according to PCWG3 criteria will be assessed every 8 weeks (±7 days) for the first 24 weeks, then every 12 weeks (±7 days) thereafter.

### Example 3: Anti-Tumor Activity of Ladiratuzumab Vedotin in Patients with Melanoma

Human patients with unresectable, locally advanced or metastatic melanoma will be treated with ladiratuzumab vedotin (LV). Patients will not have been previously treated with chemotherapy. Patients have been previously treated with anti-PD(L)1±Ipilimumab, post-treatment with a BRAF inhibitor±a MEK inhibitor.

LV will be administered by intravenous (IV) infusion at a dose of 1.0 mg/kg or 1.25 mg/kg on Day 1 of each 7-day cycle. Dosing may not exceed 200 mg per infusion, respectively. An individual's dose may be modified based upon treatment-related AEs. Tumor assessment according to RECIST v1.1 will be performed every 6 weeks (±3 days) for the first 12 months and then every 12 weeks (±7 days) thereafter. Objective responses will be confirmed with repeat scans 4-6 weeks after the first documentation of response. LV will be administered at a dose of 1.0 mg/kg or 1.25 mg/kg as a 30 minute IV infusion every 7 days.

### Example 4: Anti-Tumor Activity of Ladiratuzumab Vedotin Administered Weekly (01W) or Every Three Weeks (Q3W)

This is a global, 2-part, open-label, multi-center phase 2 study, which evaluates the safety and efficacy of weekly LV (Q1W) dosing (Days 1, 8, and 15 of every 3 week cycle), compared with LV (Q3W) dosing every 21 days, in patients with advanced solid tumors with various LIV-1 expression, including metastatic castration-resistant prostate cancer (mCRPC), and melanoma.

Specifically, human patients with (i) unresectable, locally advanced or metastatic prostate cancer or (ii) unresectable, locally advanced or metastatic melanoma are treated with ladiratuzumab vedotin (LV) in the 2-part study described hereafter. In Part A, patients will be administered LV 2.5 mg/kg IV every 3 weeks (n=up to 72); and in Part B, patients will be administered LV 1.0 or 1.25 mg/kg Q1W (n=up to 252 total). All cohorts except that of mCRPC require patients to have measurable disease per response evaluation criteria in solid tumors (RECIST) 1.1. All patients must have an Eastern Cooperative Oncology Group score of ≤1 and adequate organ function. Primary study objectives are objective tumor response rates for all cohorts plus prostate-specific antigen response rate for the mCRPC cohort. Key secondary objectives include safety, progression free survival, and overall survival.

1. A method of treating a subject having or at risk of having cancer, comprising:
administering to the subject a therapeutically effective dose of an antibody or an antigen-binding fragment thereof that specifically binds human LIV1,
wherein the antibody or antigen-binding fragment thereof comprises a heavy chain variable region (HCVR) having at least 95% identity to SEQ ID NO:1, and a light chain variable region (LCVR) having at least 95% identity to SEQ ID NO:2,
wherein the cancer is prostate cancer.

2. The method of claim 1, wherein the cancer is metastatic.

3. The method of claim 1, wherein the prostate cancer is castration resistant prostate cancer.

4. (canceled)

5. The method of claim 1, wherein the subject has not been previously treated with radioisotope therapy.

6. The method of claim 1, wherein the subject has been previously treated with no more than one line of an androgen-receptor targeted therapy.

7. The method of claim 6, wherein the androgen-receptor targeted therapy is selected from the group consisting of abiraterone acetate, enzalutamide, apalutamide, and darolutamide.

8. The method of claim 1, wherein the subject has not been previously treated with chemotherapy to treat the metastatic prostate cancer.

9. The method of claim 1, wherein the subject does not have a BRCA mutation.

10. The method of claim 1, wherein the prostate cancer is an adenocarcinoma of the prostate.

11. A method of treating a subject having or at risk of having cancer, comprising:
administering to the subject a therapeutically effective dose of an antibody or an antigen-binding fragment thereof that specifically binds human LIV1,
wherein the antibody or antigen-binding fragment thereof comprises a heavy chain variable region (HCVR) having at least 95% identity to SEQ ID NO:1, and a light chain variable region (LCVR) having at least 95% identity to SEQ ID NO:2,
wherein the cancer is melanoma.

12-20. (canceled)

21. The method of claim 1, wherein the heavy chain variable region of the antibody or antigen-binding fragment thereof comprises the three complementarity determining regions (CDRs) of SEQ ID NO:1 and the light chain variable region of the antibody or antigen-binding fragment thereof comprises the three CDRs of SEQ ID NO:2.

22. The method of claim 1, wherein the heavy chain variable region has at least 98% identity to SEQ ID NO:1 and the light chain variable region has at least 98% identity to SEQ ID NO:2.

23. (canceled)

24. The method of claim 1, wherein the heavy chain variable region comprises the sequence of SEQ ID NO:1 and the light chain variable region comprises the sequence of SEQ ID NO:2.

25. The method of claim 1, wherein the antibody or antigen-binding fragment thereof is conjugated to:
(a) monomethyl auristatin E (MMAE): 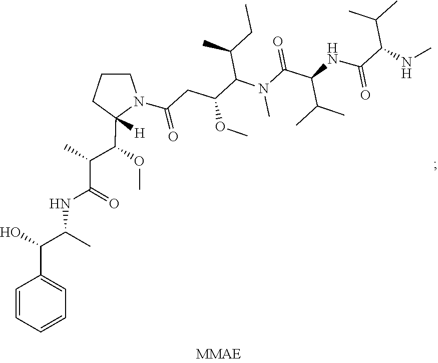 [SMILES: CC[C@H](C)C([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](NC)C(C)C)C(C)C]
(b) valine-citrulline-monomethyl auristatin E (vcMMAE):

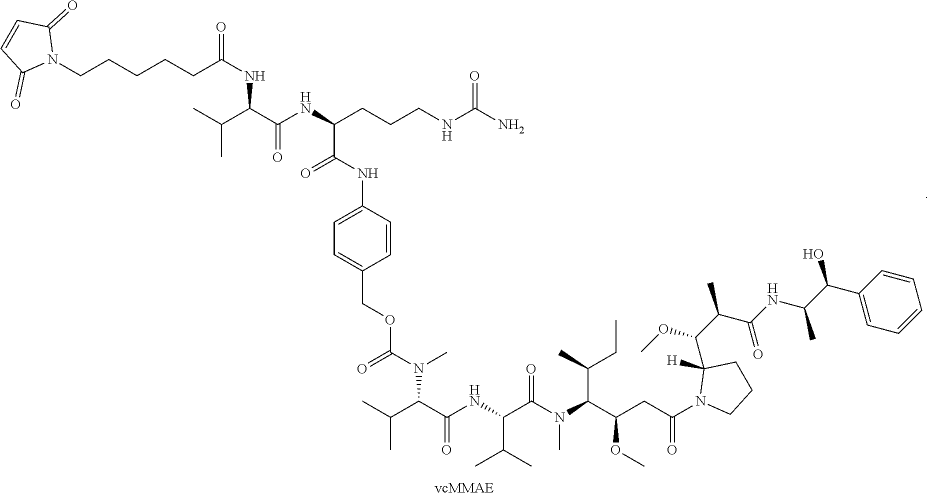 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](NC(=O)CCCCCN2C(=O)C=CC2=O)C(C)C)cc1)C(C)C]

26. (canceled)

27. The method of claim 25, wherein a vcMMAE to antibody or antigen-binding fragment thereof ratio is from about 1 to about 8.

28. (canceled)

29. The method of claim 1, wherein the dose administered is less than about 200 mg of the antibody or antigen-binding fragment thereof per treatment cycle.

30. The method of claim 1, wherein the dose is about 1.0 mg/kg or about 1.25 mg/kg of body weight of the subject.

31-33. (canceled)

34. The method of claim 1, wherein the treatment cycle is a Q1W treatment cycle.

35. The method of claim 1, wherein the subject has been previously treated with one or more therapeutic agents and did not respond to the treatment, relapsed after the treatment, or has experienced disease progression during treatment, wherein the one or more therapeutic agents is not the antibody or antigen-binding fragment thereof.

36-37. (canceled)

38. The method of claim 1, wherein the cancer is an advanced stage cancer.

39. (canceled)

40. The method of claim 1, wherein the cancer is recurrent cancer.

41. The method of claim 1, wherein the cancer is unresectable.

42-43. (canceled)

44. The method of claim 1, wherein one or more therapeutic effects in the subject is improved after administration of the antibody or antigen-binding fragment thereof relative to a baseline, wherein the one or more therapeutic effects is selected from the group consisting of: size of a tumor derived from the cancer, objective response rate, duration of response, time to response, progression free survival, overall survival, prostate-specific antigen (PSA) level, PSA duration of response, and PSA-PFS.

45-57. (canceled)

58. The method of claim 1, wherein the route of administration for the antibody or antigen-binding fragment thereof is intravenous infusion.

59. The method of claim 1, wherein the antibody or antigen-binding fragment thereof is administered as a monotherapy or in combination with a checkpoint inhibitor.

60. (canceled)

61. The method of claim 59 wherein the checkpoint inhibitor is an anti-PD-1 antibody, an anti-PD-L1 antibody, an anti-CTLA4 antibody, B7-DC-Fc, LAG3, or TIM3.

62. The method of claim 59 wherein the checkpoint inhibitor is selected from the group consisting of MEDI0680, AMP-224, nivolumab, pembrolizumab, pidilizumab, MEDI4736, MPDL3280A, ipilimumab and tremelimumab.

63-65. (canceled)

66. A kit comprising:
(a) a dosage ranging from about 0.5 mg/kg to about 2.8 mg/kg of an antibody or antigen-binding fragment thereof that binds LIV1; and
(b) instructions for using the antibody or antigen-binding fragment thereof according to the method of claim 1.
